(19) United States
(12) Patent Application Publication
(10) Pub. No.: US 2005/0123977 A1
(43) Pub. Date: Jun. 9, 2005

(54) Assay and treatment

(75) Inventor: Xin Lu, London (GB)

Correspondence Address: KLARQUIST SPARKMAN, LLP, 121 SW SALMON STREET, SUITE 1600, PORTLAND, OR, 97204, US

(73) Assignee: Ludwig Institute for Cancer Research

(21) Appl. No.: 11/004,749

(22) Filed: Dec. 3, 2004

Related U.S. Application Data
(60) Provisional application No. 60/554,686, filed on Mar. 19, 2004.

(30) Foreign Application Priority Data
Feb. 27, 2004 (GB) GB0404350.1
Dec. 4, 2003 (GB) GB0328047.6

(51) Int. Cl. C12Q 1/68; G01N 33/574; C07H 21/04; C12N 9/64; C07K 16/40

(52) U.S. Cl. 435/6; 435/7.23; 435/455; 435/320.1; 435/325; 435/226; 536/23.2; 530/388.8

(57) ABSTRACT

We describe screening methods for agents which modulate the interaction of p53 activator/inhibitor binding proteins with polymorphic p53 polypeptide variants, cells expressing combinations of said activator/inhibitor proteins and p53 variants; diagnostic assays to determine the genotype of an individual with respect to said p53 polymorphism; and therapeutic compositions for use in the treatment of conditions which would benefit from a stimulation of apoptosis.

### CROSS-REFERENCE TO RELATED APPLICATIONS

This application claims priority to U.S. Provisional Application No. 60/554,686 filed Mar. 19, 2004, to Great Britain Application No. GB0404350.1 filed Feb. 27, 2004, and to Great Britain Application No. GB0328047.6 filed Dec. 4, 2003.

### FIELD

The disclosure relates to screening methods for agents that modulate the interaction of p53 activator/inhibitor binding proteins with polymorphic p53 polypeptide variants; diagnostic assays to determine the genotype of an individual with respect to said p53 polymorphism; and including compositions comprising nucleic acid molecules encoding p53, or variants thereof, in combination with p53 pro-apoptotic polypeptides and optionally therapeutic agents

### BACKGROUND

Tumor suppressor genes encode proteins which function to inhibit cell growth or division and are therefore important with respect to maintaining proliferation, growth and differentiation of normal cells. Mutations in tumour suppressor genes result in abnormal cell-cycle progression whereby the normal cell-cycle check points which arrest the cell-cycle, when, for example, DNA is damaged, are ignored and damaged cells divide uncontrollably. The products of tumour suppressor genes function in all parts of the cell (such as the cell surface, cytoplasm, nucleus) to prevent the passage of damaged cells through the cell-cycle (G1, S, G2, M and cytokinesis).

Arguably the tumor suppressor gene which has been the subject of the most intense research is p53. p53 encodes a protein which functions as a transcription factor and is a key regulator of the cell division cycle. It was discovered as a protein shown to bind with affinity to the SV40 large T antigen. The p53 gene encodes a 393 amino acid polypeptide with a molecular weight of 53 kDa.

### SUMMARY

A large number of studies have been carried out to investigate the relationship between a common polymorphism of p53 at codon 72 (Arg/Pro) and cancer susceptibility. However the existing conclusions are controversial due to a lack of understanding of how and why the two p53 polymorphism variants function differently. It is shown herein that the apoptotic function of the two polymorphic p53 variants depends on their ability to interact with the ASPP family of proteins, as described in WO02/12325. ASPP1 and ASPP2 selectively interact with and stimulate the apoptotic function of p53Pro72 while iASPP selectively bind and inhibit the apoptotic function of p53Pro72.

Provided herein is a screening method for the identification of agents which modulate the interaction of a polypeptide encoded by a nucleic acid molecule selected from the group consisting of:
a) a polypeptide encoded by a nucleic acid molecule comprising a nucleic acid sequence as represented in FIG. 6 (SEQ ID NO: 1);
b) a polypeptide encoded by a nucleic acid molecule which hybridises to the nucleic acid molecule in (a) and which inhibits the apoptotic activity of p53;
c) a polypeptide encoded by a nucleic acid molecule consisting of a nucleic acid sequence which is degenerate as a result of the genetic code to a nucleic acid molecule as defined in (a) and (b);
   with a p53 polymorphic polypeptide variant wherein said variant is modified by substitution of an amino acid residue encoded by codon 72 of the nucleic acid sequence as represented in FIG. 7 (SEQ ID NO: 2) comprising:
i) forming a preparation comprising said polypeptide and said p53 variant;
ii) adding at least one candidate agent to be tested; and
iii) determining the effect, or not, of said agent on the interaction of said polypeptide with said p53 variant.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (Allows Sequences that Share at Least 80% Identity to Hybridize)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (Allows Sequences that Share at Least 50% Identity to Hybridize)

Hybridization: 6×SSC at RT to 55° C. for 16-20 hours

Wash at least twice: 2×-3×SSC at RT to 55° C. for 20-30 minutes each.

Nucleic acid molecules which hybridise to any nucleic acid molecule disclosed herein (such as SEQ ID NO: 1, 2, 5 or 6) can do so with very high, high, or low stringency.

In one particular example, the polypeptide is encoded by a nucleic acid molecule consisting of a nucleic acid sequence as represented by FIG. 6 (SEQ ID NO: 1).

In a yet another example, said polypeptide is represented by the amino acid sequence as shown in FIG. 8 (SEQ ID NO: 4), or a variant polypeptide wherein said variant polypeptide is modified by addition, deletion or substitution of at least one amino acid residue which varies with respect to the amino acid sequence shown in FIG. 8 (SEQ ID NO: 4). Ideally, the variant polypeptide retains the activity of said polypeptide or has enhanced activity.

A variant polypeptide may differ in amino acid sequence by one or more substitutions, additions, deletions, truncations which may be present in any combination. Variants include those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and asparatic acid; c) asparagine and glutamine d) arginine and lysine; e) isoleucine, leucine, methionine and valine and f) phenylalanine, tyrosine and tryptophan.

A polypeptide is a variant wherein one or more amino acid residues are substituted with conserved or non-conserved amino acid residues, or one in which one or more amino acid residues includes a substituent group. Conservative substitutions are the replacements, one for another, among the aliphatic amino acids Ala, Val, Leu and Ile; interchange of the hydroxl residues Ser and Thr; exchange of the acidic residues Asp and Glu; substitution between amide residues Asn and Gln; exchange of the basic residues Lys and Arg; and replacements among aromatic residues Phe and Tyr.

In addition, the disclosure features polypeptide sequences having at least 75% identity with the polypeptide sequence as herein disclosed, or fragments and functionally equivalent polypeptides thereof. In one embodiment, the polypeptides have at least 85% identity, such as at least 90% identity, such as at least 95% identity, such as at least 97% identity, and even at least 99% identity with the amino acid sequences illustrated herein.

In a one example, said p53 variant varies at codon 72 wherein said codon encodes a proline amino acid residue.

Exemplary agents include, but are not limited to, an antagonist and a polypeptide. In one example, said polypeptide is an antibody or active binding part thereof, such as a monoclonal antibody.

Antibodies or immunoglobulins (Ig) are a class of structurally related proteins consisting of two pairs of polypeptide chains, one pair of light (L) (low molecular weight) chain (κ or λ), and one pair of heavy (H) chains (γ, α, μ, δ and ε), all four linked together by disulphide bonds. Both H and L chains have regions that contribute to the binding of antigen and that are highly variable from one Ig molecule to another. In addition, H and L chains contain regions that are non-variable or constant. The L chains consist of two domains. The carboxy-terminal domain is essentially identical among L chains of a given type and is referred to as the “constant” (C) region. The amino terminal domain varies from L chain to L chain and contributes to the binding site of the antibody. Because of its variability, it is referred to as the “variable” (V) region. The variable region contains complementary determining regions or CDR's which form an antigen binding pocket. The binding pockets comprise H and L variable regions which contribute to antigen recognition. It is possible to create single variable regions, so called single chain antibody variable region fragments (scFv's). If a hybridoma exists for a specific monoclonal antibody it is well within the knowledge of the skilled person to isolate scFv's from mRNA extracted from said hybridoma via RT PCR. Alternatively, phage display screening can be undertaken to identify clones expressing scFv's.

Alternatively said fragments are “domain antibody fragments”. Domain antibodies are the smallest binding part of an antibody (approximately 13 kDa). Examples of this technology is disclosed in U.S. Pat. No. 6,248,516, U.S. Pat. No. 6,291,158, U.S. Pat. No. 6,127,197 and EP0368684 which are all incorporated by reference in their entirety.

In one example, said antibody fragment is a single chain antibody variable region fragment. In another example, the antibody is a humanised or chimeric antibody. A chimeric antibody is produced by recombinant methods to contain the variable region of an antibody with an invariant or constant region of a human antibody. A humanised antibody is produced by recombinant methods to combine the complementary determining regions (CDRs) of an antibody with both the constant (C) regions and the framework regions from the variable (V) regions of a human antibody.

Chimeric antibodies are recombinant antibodies in which all of the V-regions of a mouse or rat antibody are combined with human antibody C-regions. Humanised antibodies are recombinant hybrid antibodies which fuse the complimentarity determining regions from a rodent antibody V-region with the framework regions from the human antibody V-regions. The C-regions from the human antibody are also used. The complimentarity determining regions (CDRs) are the regions within the N-terminal domain of both the heavy and light chain of the antibody to where the majority of the variation of the V-region is restricted. These regions form loops at the surface of the antibody molecule. These loops provide the binding surface between the antibody and antigen.

Antibodies from non-human animals provoke an immune response to the foreign antibody and its removal from the circulation. Both chimeric and humanised antibodies have reduced antigenicity when injected to a human subject because there is a reduced amount of rodent (that is, foreign) antibody within the recombinant hybrid antibody, while the human antibody regions do not elicit an immune response. This results in a weaker immune response and a decrease in the clearance of the antibody. This is clearly desirable when using therapeutic antibodies in the treatment of human diseases. Humanised antibodies are designed to have less “foreign” antibody regions and are therefore thought to be less immunogenic than chimeric antibodies.

In one example, said agent is a peptide, such as a modified peptide.

It will be apparent to one skilled in the art that modification to the amino acid sequence of peptides which modulate the interaction of iASPP and p53pro72 could enhance the binding and/or stability of the peptide with respect to its target sequence. In addition, modification of the peptide may also increase the in vivo stability of the peptide thereby reducing the effective amount of peptide necessary to induce apoptosis. This would advantageously reduce undesirable side effects which may result in vivo.

Modifications include, by example and not by way of limitation, acetylation and amidation.

In one example, said peptide is acetylated, such as acetylation of the amino terminus of said peptide. In another example, said peptide is amidated, such as at the carboxyl-terminus of said peptide. In yet another example, said peptide is modified by both acetylation and amidation.

The modification can include the use of modified amino acids in the production of recombinant or synthetic forms of peptides. It will be apparent to one skilled in the art that modified amino acids include, by way of example and not by way of limitation, 4-hydroxyproline, 5-hydroxylysine, N⁶-acetyllysine, N⁶-methyllysine, N⁶,N⁶-dimethyllysine, N⁶,N⁶,N⁶-trimethyllysine, cyclohexyalanine, D-amino acids, ornithine. Other modifications include amino acids with a C₂, C₃ or C₄ alkyl R group optionally substituted by 1, 2 or 3 substituents selected from halo (such as F, Br, I), hydroxy or C₁-C₄ alkoxy.

Alternatively, peptides can modified by, for example, cyclisation. Cyclisation is known in the art, (see Scott et al., Chem. Biol. (2001), 8:801-815; Gellerman et al., J. Peptide Res (2001), 57: 277-291; Dutta et al., J. Peptide Res (2000), 8: 398-412; Ngoka and Gross J Amer Soc Mass Spec (1999), 10:360-363. In one particular example, the peptides disclosed herein are modified by cyclisation.

In another example, said agent is an aptamer.

Nucleic acids have both linear sequence structure and a three dimensional structure which in part is determined by the linear sequence and also the environment in which these molecules are located. Conventional therapeutic molecules are small molecules, for example, peptides, polypeptides, or antibodies, which bind target molecules to produce an agonistic or antagonistic effects. It has become apparent that nucleic acid molecules also have potential with respect to providing agents with the requisite binding properties which may have therapeutic utility. These nucleic acid molecules are typically referred to as aptamers. Aptamers are small, usually stabilised, nucleic acid molecules, which comprise a binding domain for a target molecule. A screening method to identify aptamers is described in U.S. Pat. No. 5,270,163(herein incorporated by reference). Aptamers are typically oligonucleotides which may be single stranded oligodeoxynucleotides, oligoribonucleotides, or modified oligodeoxynucleotide or oligoribonucleotides.

The term “modified” encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2;azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include, by example and not by way of limitation, alkylated purines and/or pyrimidines; acylated purines and/or pyrimidines; or other heterocycles. These classes of pyrimidines and purines are known in the art and include, pseudoisocytosine; N4, N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2 methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methylcytosine.

Aptamers can be synthesised using conventional phosphodiester linked nucleotides and synthesised using standard solid or solution phase synthesis techniques which are known in the art. Linkages between nucleotides may use alternative linking molecules. For example, linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through —O— or —S—. The binding of aptamers to a target polypeptide is readily tested by assays herein disclosed.

The disclosed method can further include a step wherein said candidate agent is tested for activity with respect to a second different p53 polymorphic polypeptide variant, for example when said p53 variant is modified by substitution of an amino acid residue encoded by codon 72 of the nucleic acid sequence as represented in FIG. 7 (SEQ ID NO: 2).

In one example, said p53 variant varies at codon 72 wherein said codon encodes an arginine amino acid residue.

In a another example, said preparation comprises a cell transfected with a first nucleic acid molecule selected from the group consisting of:
i) a nucleic acid molecule comprising a nucleic acid sequence as represented in FIG. 6 (SEQ ID NO: 1);
ii) a nucleic acid molecule which hybridises to the nucleic acid molecule in (i) and which encodes a polypeptide which inhibits the apoptotic activity of p53;
iii) a nucleic acid molecule consisting of a nucleic acid sequence which is degenerate as a result of the genetic code to a nucleic acid molecule as defined in (i) and (ii); and a second nucleic acid molecule which encodes a p53 polymorphic polypeptide variant wherein said variant is modified by substitution of an amino acid residue encoded by codon 72 of the nucleic acid sequence as represented in FIG. 7 (SEQ ID NO: 2).

In one example, said p53 variant varies at codon 72 wherein said codon encodes a proline amino acid residue.

According to a further aspect of the disclosure, there is provided a cell transfected with a first nucleic acid molecule selected from the group consisting of:
i) a nucleic acid molecule comprising a nucleic acid sequence as represented in FIG. 6 (SEQ ID NO: 1);
ii) a nucleic acid molecule which hybridises to the nucleic acid molecule in (i) and which encodes a polypeptide which inhibits the apoptotic activity of p53;
iii) a nucleic acid molecule consisting of a nucleic acid sequence which is degenerate as a result of the genetic code to a nucleic acid molecule as defined in (i) and (ii); and a second nucleic acid molecule which encodes a p53 polymorphic polypeptide variant wherein said variant is modified by substitution of an amino acid residue encoded by codon 72 of the nucleic acid sequence as represented in FIG. 7 (SEQ ID NO: 2).

In one embodiment of the disclosure said cell is transfected with a second nucleic acid molecule which encodes a p53 variant which varies at codon 72 wherein said codon encodes a proline amino acid residue.

In one embodiment of the disclosure said cell is a cancer cell.

According to a further aspect of the disclosure there is provided a screening method for the identification of agents which modulate the interaction of a polypeptide encoded by a nucleic acid molecule selected from the group consisting of:
a) a polypeptide encoded by a nucleic acid molecule comprising a nucleic acid sequence as represented in FIGS. 9a or 9b (SEQ ID NOS: 5 or 6);
b) a polypeptide encoded by a nucleic acid molecule which hybridises to the nucleic acid molecule in (a) and which stimulates the apoptotic activity of p53;
c) a polypeptide encoded by a nucleic acid molecule consisting of a nucleic acid sequence which is degenerate as a result of the genetic code to a nucleic acid molecule as defined in (a) and (b);
   with a p53 polymorphic polypeptide variant wherein said variant is modified by substitution of an amino acid residue encoded by codon 72 of the nucleic acid sequence as represented in FIG. 7 (SEQ ID NO: 2) comprising:
   i) forming a preparation comprising said polypeptide and said p53 variant;
   ii) adding at least one candidate agent to be tested; and
   iii) determining the effect, or not, of said agent on the interaction of said polypeptide with said p53 variant.

In a further example, said polypeptide is encoded by a nucleic acid molecule consisting of a nucleic acid sequence as represented by FIGS. 9a or 9b (SEQ ID NO: 5 or 6).

In a yet another example, said polypeptide is represented by the amino acid sequence as shown in FIGS. 10a or 10b (SEQ ID NO: 7 or 8), or a variant polypeptide wherein said variant polypeptide is modified by addition, deletion or substitution of at least one amino acid residue which varies with respect to the amino acid sequence shown in FIGS. 10a or 10b (SEQ ID NO: 7 or 8). Ideally, said variant polypeptide retains the activity of said polypeptide or has enhanced activity.

In one example, said agent is an agonist that mimics or augments the pro-apoptotic activity of said polypeptides or polypeptide variants.

It will be apparent that agents can either mimic the pro-apoptotic effect of ASPP1 or ASPP2 or enhances the effect of these pro-apoptotic polypeptides.

In a one method of the disclosure, said agent is a polypeptide, antibody, or peptides or aptamers as described previously.

In a further example, said preparation comprises a cell transfected with a first nucleic acid molecule selected from the group consisting of:
i) a nucleic acid molecule comprising a nucleic acid sequence as represented in FIGS. 9a or 9b (SEQ ID NO: 5 or 6);
ii) a nucleic acid molecule which hybridises to the nucleic acid molecule in (i) and which encodes a polypeptide which stimulates the apoptotic activity of p53;
iii) a nucleic acid molecule consisting of a nucleic acid sequence which is degenerate as a result of the genetic code to a nucleic acid molecule as defined in (i) and (ii); and a second nucleic acid molecule which encodes a p53 polymorphic polypeptide variant wherein said variant is modified by substitution of an amino acid residue encoded by codon 72 of the nucleic acid sequence as represented in FIG. 7 (SEQ ID NO: 2).

In one example, said p53 variant varies at codon 72 wherein said codon encodes a proline amino acid residue.

According to a flurther aspect of the disclosure there is provided a cell transfected with a first nucleic acid molecule selected from the group consisting of:
i) a nucleic acid molecule comprising a nucleic acid sequence as represented in FIGS. 9a or 9b (SEQ ID NO: 5 or 6);
ii) a nucleic acid molecule which hybridises to the nucleic acid molecule in (i) and which encodes a polypeptide which stimulates the apoptotic activity of p53;
iii) a nucleic acid molecule consisting of a nucleic acid sequence which is degenerate as a result of the genetic code to a nucleic acid molecule as defined in (i) and (ii); and a second nucleic acid molecule which encodes a p53 polymorphic polypeptide variant wherein said variant is modified by substitution of an amino acid residue encoded by codon 72 of the nucleic acid sequence as represented in FIG. 7 (SEQ ID NO: 2).

In one example, said cell is transfected with a second nucleic acid molecule which encodes a p53 variant which varies at codon 72 wherein said codon encodes a proline amino acid residue.

In one example, said cell is a cancer cell.

According to a further aspect of the disclosure there is provided a method of diagnosis, of an animal, such as a human, which determines both the p53 genotype and the expression of ASPP1 and/or ASPP2 and/or iASPP in a tissue sample.

In one example, said method of diagnosis comprises:
i) providing a cell or tissue sample to be tested;
ii) determining the p53 genotype of said animal from analysis of said sample; and optionally
iii) determining the expression of at least one gene product encoded by the genes ASPP1, ASPP2 or iASPP.

In one example, said p53 genotype is p53pro72 or p53arg72.

In one example, said method of diagnosis includes a further step wherein the treatment regime of said animal/human is determined by the combination of p53 genotype and the expression status of at least one gene product encoded by ASPP1, ASPP2 or iASPP.

In a particular example, said cell/tissue sample is a breast sample.

According to an aspect of the disclosure there is provided a composition comprising at least one nucleic acid molecule wherein said nucleic acid molecule encodes a p53 polypeptide, or sequence variant thereof, and at least one member of the ASPP family of pro-apoptotic polypeptides.

According to a further aspect of the disclosure there is provided a composition comprising a first nucleic acid molecule selected from the group consisting of:
i) a nucleic acid molecule comprising a nucleic acid sequence as represented in FIGS. 9a or 9b (SEQ ID NO: 5 or 6);
ii) a nucleic acid molecule which hybridises to the nucleic acid molecule in (i) and which encodes a polypeptide which stimulates the apoptotic activity of p53;
iii) a nucleic acid molecule consisting of a nucleic acid sequence which is degenerate as a result of the genetic code to a nucleic acid molecule as defined in (i) and (ii); and a second nucleic acid molecule comprising a nucleic acid sequence selected from the group consisting of:
iv) a nucleic acid molecule consisting of a nucleic acid sequence as represented in FIG. 7 (SEQ ID NO: 2);
v) a nucleic acid molecule consisting of a nucleic acid sequence which hybridises to the nucleic acid molecule in (iv) and which encodes a polypeptide with the specific activity associated with p53;
vi) a nucleic acid molecule consisting of a nucleic acid sequence which is degenerate as a result of the genetic code to a nucleic acid molecule as defined in (iv) and (v); wherein said nucleic acid molecules are operably linked to a promoter sequence which facilitates the expression of said first and second nucleic acid molecules.

In one example, said p53 polypeptide is p53pro72.

In one example, said nucleic acid molecules are part of an expression vector, such as an expression vector adapted for eukaryotic gene expression.

Typically said adaptation includes, by example and not by way of limitation, the provision of transcription control sequences (promoter sequences) which mediate cell/tissue specific expression. These promoter sequences may be cell/tissue specific, inducible or constitutive.

“Promoter” is an art recognized term and, for the sake of clarity, includes the following features which are provided by example only, and not by way of limitation. Enhancer elements are cis acting nucleic acid sequences often found 5′ to the transcription initiation site of a gene (enhancers can also be found 3′ to a gene sequence or even located in intronic sequences). Enhancers function to increase the rate of transcription of the gene to which the enhancer is linked. Enhancer activity is responsive to trans acting transcription factors (polypeptides) which have been shown to bind specifically to enhancer elements. The binding/activity of transcription factors (please see Eukaryotic Transcription Factors, by David S Latchman, Academic Press Ltd, San Diego) is responsive to a number of physiological/environmental cues which include, by example and not by way of limitation, intermediary metabolites (such as glucose, lipids), environmental effectors (such as light, heat,).

Promoter elements also include so called TATA box and RNA polymerase initiation selection sequences which fumction to select a site of transcription initiation. These sequences also bind polypeptides which function, inter alia, to facilitate transcription initiation selection by RNA polymerase.

Adaptations also include the provision of selectable markers and autonomous replication sequences which facilitate the maintenance of said vector in either the eukaryotic cell or prokaryotic host. Vectors which are maintained autonomously are referred to as episomal vectors. Episomal vectors are desirable since these molecules can incorporate large DNA fragments (30-50 kb DNA). Episomal vectors of this type are described in WO98/07876.

Adaptations which facilitate the expression of vector encoded genes include the provision of transcription termination/polyadenylation sequences. This also includes the provision of internal ribosome entry sites (IRES) which function to maximise expression of vector encoded genes arranged in bi-cistronic or multi-cistronic expression cassettes. Expression control sequences also include so-called Locus Control Regions (LCRs). These are regulatory elements which confer position-independent, copy number-dependent expression to linked genes when assayed as transgenic constructs. LCRs include regulatory elements that insulate transgenes from the silencing effects of adjacent heterochromatin, Grosveld et al., Cell (1987), 51: 975-985.

There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc. (1994).

The use of viruses or “viral vectors” as therapeutic agents is well known in the art. Additionally, a number of viruses are commonly used as vectors for the delivery of exogenous genes. Commonly employed vectors include recombinantly modified enveloped or non-enveloped DNA and RNA viruses, such as baculoviridiae, parvoviridiae, picornoviridiae, herpesveridiae, poxviridae, adenoviridiae, or picornnaviridiae. Chimeric vectors may also be employed which exploit advantageous elements of each of the parent vector properties (See e.g., Feng, et al.(1997) Nature Biotechnology 15:866-870). Such viral vectors may be wild-type or may be modified by recombinant DNA techniques to be replication deficient, conditionally replicating or replication competent.

Exemplary vectors are derived from the adenoviral, adeno-associated viral and retroviral genomes. In one example, the vectors are derived from the human adenovirus genome, such as human adenovirus serotypes 2 or 5. The replicative capacity of such vectors may be attenuated (to the point of being considered “replication deficient”) by modifications or deletions in the E1a and/or E1b coding regions. Other modifications to the viral genome to achieve particular expression characteristics or permit repeat administration or lower immune response are preferred. In one example, the vector is replication deficient vector adenoviral vector encoding the p53 tumor suppressor gene A/C/N/53 as described in Gregory, et al., U.S. Pat. No. 5,932,210 issued Aug. 3, 1999 (the entire teaching of which is herein incorporated by reference).

Alternatively, the viral vectors may be conditionally replicating or replication competent. Conditionally replicating viral vectors are used to achieve selective expression in particular cell types while avoiding untoward broad spectrum infection. Examples of conditionally replicating vectors are described in Pennisi, E. (1996) Science 274:342-343; Russell, and S. J. (1994) Eur. J. of Cancer 30A(8):1165-1171. Additional examples of selectively replicating vectors include those vectors wherein a gene essential for replication of the virus is under control of a promoter which is active only in a particular cell type or cell state such that in the absence of expression of such gene, the virus will not replicate. Examples of such vectors are described in Henderson, et al., U.S. Pat. No. 5,698,443 issued Dec. 16, 1997 and Henderson, et al., U.S. Pat. No. 5,871,726 issued Feb. 16, 1999 the entire teachings of which are herein incorporated by reference.

Additionally, the viral genome may be modified to include inducible promoters which achieve replication or expression only under certain conditions. Examples of inducible promoters are known in the scientific literature (See for example Yoshida and Hamada (1997) Biochem. Biophys. Res. Comm. 230:426-430; Iida, et al. (1996) J. Virol. 70(9):6054-6059; Hwang, et al.(l997) J. Virol 71(9):7128-7131; Lee, et al. (1997) Mol. Cell. Biol. 17(9):5097-5105; and Dreher, et al.(1997) J. Biol. Chem 272(46); 29364-29371.

The viruses may also be designed to be selectively replicating viruses. Exemplary selectively replicating viruses are described in Ramachandra, et al. PCT International Publication No. WO 00/22137, International Application No. PCT/US99/21452 published Apr. 20, 2000 and Howe, J., PCT International Publication No. WO WO0022136, International Application No. PCT/US99/21451 published Apr. 20, 2000. It has been demonstrated that viruses which are attenuated for replication are also useful in the therapeutic arena. For example the adenovirus dl1520 containing a specific deletion in the E1b5SK gene (Barker and Berk (1987) Virology 156: 107) has been used with therapeutic effect in human beings. Such vectors are also described in McCormick (U.S. Pat. No. 5,677,178 issued Oct. 14, 1997) and McCormick, U.S. Pat. No 5,846,945 issued Dec. 8, 1998. The method of the present disclosure may also be used in combination with the administration of such vectors to minimize the pre-existing or induced humoral immune response to such vectors.

It may be valuable in some instances to utilize or design vectors to achieve introduction of the exogenous transgene in a particular cell type. Certain vectors exhibit a natural tropism for certain tissue types. For example, vectors derived from the genus herpesviridiae have been shown to have preferential infection of neuronal cells. Examples of recombinantly modified herpesviridiae vectors are disclosed in U.S. Pat. No. 5,328,688 issued Jul. 12, 1994. Cell type specificity or cell type targeting may also be achieved in vectors derived from viruses having characteristically broad infectivities by the modification of the viral envelope proteins. For example, cell targeting has been achieved with adenovirus vectors by selective modification of the viral genome knob and fiber coding sequences to achieve expression of modified knob and fiber domains having specific interaction with unique cell surface receptors. Examples of such modifications are described in Wickham, et al. (1997) J. Virol 71(11):8221-8229 (incorporation of RGD peptides into adenoviral fiber proteins); Arnberg, et al.(1997) Virology 227:239-244 (modification of adenoviral fiber genes to achieve tropism to the eye and genital tract); Harris and Lemoine (1996) TIG 12(10):400-405; Stevenson, et al.(1997) J. Virol. 71(6):4782-4790; Michael, et al.(1995) Gene Therapy 2:660-668 (incorporation of gastrin releasing peptide fragment into adenovirus fiber protein); and Ohno, et al.(1997) Nature Biotechnology 15:763-767 (incorporation of Protein A-IgG binding domain into Sindbis virus). Other methods of cell specific targeting have been achieved by the conjugation of antibodies or antibody fragments to the envelope proteins (see, e.g. Michael, et al. (1993) J. Biol. Chem 268:6866-6869, Watkins, et al. (1997) Gene Therapy 4:1004-1012; Douglas, et al.(1996) Nature Biotechnology 14: 1574-1578. Alternatively, particularly moieties may be conjugated to the viral surface to achieve targeting (See, e.g. Nilson, et al. (1996) Gene Therapy 3:280-286 (conjugation of EGF to retroviral proteins)). Additionally, the virally encoded therapeutic transgene also be under control of a tissue specific-promoter region allowing expression of the transgene preferentially in particular cell types.

In one example, said composition is a therapeutic composition.

When administered, the therapeutic compositions of the present disclosure are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents, such as chemotherapeutic agents.

The therapeutics of the disclosure can be administered by any conventional route, including injection or by gradual infusion over time. The administration may, for example, be oral, intravenous, intraperitoneal, intramuscular, intracavity, subcutaneous, or transdermal.

The compositions of the disclosure are administered in effective amounts. An “effective amount” is that amount of a composition that alone, or together with further doses, produces the desired response. In the case of treating a particular disease, such as cancer, the desired response is inhibiting the progression of the disease. This may involve only slowing the progression of the disease temporarily, although it can involve halting the progression of the disease permanently. This can be monitored by routine methods or can be monitored according to diagnostic methods discussed herein.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. In a particular example, a maximum dose of the individual components or combinations is used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods can be sterile and contain an effective amount of nucleic acid for producing the desired response in a unit of weight or volume suitable for administration to a patient. The response can, for example, be measured by determining regression of a tumour, decrease of disease symptoms, modulation of apoptosis, etc.

The doses of nucleic acid administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, doses of nucleic acids of between 1 ng and 0.1 mg generally will be formulated and administered according to standard procedures. Other protocols for the administration of compositions will be known to one of ordinary skill in the art, in which the dose amount, schedule of injections, sites of injections, mode of administration (such as intra-tumoral) and the like vary from the foregoing. Administration of compositions to mammals other than humans, such as for testing purposes or veterinary therapeutic purposes, is carried out under substantially the same conditions as described above. A subject, as used herein, is a mammal, such as a human, and including a non-human primate, cow, horse, pig, sheep, goat, dog, cat or rodent.

When administered, the pharmaceutical preparations disclosed herein are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the disclosure. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Compositions may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present disclosure, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions suitable for parenteral administration conveniently comprise a sterile aqueous or non-aqueous preparation of nucleic acids, which is ideally isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

In one example, said composition fIurther comprises at least one further therapeutic agent, such as a chemotherapeutic agent. Exemplary agents include, but are not limited to: cisplatin; carboplatin; cyclosphosphamide; melphalan; carmusline; methotrexate; 5-fluorouracil; cytarabine; mercaptopurine; daunorubicin; doxorubicin; epirubicin; vinblastine; vincristine; dactinomycin; mitomycin C; taxol; L-asparaginase; G-CSF; etoposide; colchicine; derferoxamine mesylate; and camptothecin.

According to an aspect of the disclosure there is provided a method of treatment of an animal, such as a human, suffering from a condition which would benefit from the stimulation of apoptosis comprising administering at least one nucleic acid molecule comprising a nucleic acid sequence which encodes a p53 polypeptide, or variant thereof, and a polypeptide which stimulates the proapoptotic activity of said p53 polypeptide wherein said polypeptide is represented by the nucleic acid sequence as shown in FIG. 9a or FIG. 9b, or a nucleic molecule which hybridises to these sequences under stringent hybridisation conditions.

In one example, said variant p53 polypeptide is p53arg72.

In another example, said variant p53 polypeptide is p53pro72.

According to a further aspect of the disclosure there is provided a method for the diagnosis and treatment of an animal, such as a human, suffering from a condition which would benefit from the stimulation of apoptosis comprising:
i) providing a cell or tissue sample to be tested;
ii) determining the p53 genotype of said animal from analysis of said sample; and
iii) administering a composition according to the disclosure.

In one example, said p53 genotype is p53arg72 or p53pro72.

In a further example, said composition comprises at least one nucleic acid molecule comprising a nucleic acid sequence encoding a p53pro72 polypeptide and a nucleic acid sequence encoding a polypeptide which stimulates the proapoptotic activity of said p53 polypeptide wherein said polypeptide is represented by the nucleic acid sequence as shown in FIG. 9a or FIG. 9b (SEQ ID NO: 5 or 6), or a nucleic molecule which hybridises to these sequences.

In one example, said condition is cancer.

According to a further aspect of the disclosure there is provided a composition comprising at least one nucleic acid molecule comprising a nucleic acid sequence which encodes a p53 polypeptide, or variant thereof, and a nucleic acid molecule which encodes an agent which antagonises the activity of a polypeptide encoded by a nucleic acid molecule comprising a nucleic acid sequence as represented by FIG. 6 (SEQ ID NO: 1), or a nucleic acid molecule which hybridises these sequences under stringent hybridisation conditions.

In one example, said p53 variant is p53arg72.

In another example, said p53 variant is p53pro72.

In one example, said agent is an antisense RNA or an RNAi molecule.

In a further embodiment of the disclosure said agent is an antibody, or active binding fragment thereof as herein described, and which binds the polypeptide as represented by the amino acid sequence shown in FIG. 8 (SEQ ID NO: 4).

In a still further embodiment of the disclosure said composition comprises at least one further therapeutic agent, such as a chemotherapeutic agent. Exemplary agents include, but are not limited to: cisplatin; carboplatin; cyclosphosphamide; melphalan; carmusline; methotrexate; 5-fluorouracil; cytarabine; mercaptopurine; daunorubicin; doxorubicin; epirubicin; vinblastine; vincristine; dactinomycin; mitomycin C; taxol; L-asparaginase; G-CSF; etoposide; colchicine; derferoxamine mesylate; and camptothecin.

According to a further aspect of the disclosure there is provided a method for the treatment of an animal which would benefit from a stimulation of apoptosis comprising administering a composition according to the disclosure.

According to a yet further aspect of the disclosure there is provided a method for the diagnosis and treatment of an animal, such as a human, comprising:
i) providing a cell or tissue sample to be tested;
ii) determining the p53 genotype of said animal from analysis of said sample; and
iii) administering a composition according to the disclosure.

In one example, said p53 genotype is p53arg72.

In another example, said p53 genotype is p53pro72.

The singular forms “a,” “an,” and “the” refer to one or more than one, unless the context clearly dictates otherwise. For example, the term “comprising a nucleic acid molecule” includes single or plural nucleic acids and is considered equivalent to the phrase “comprising at least one nucleic acid molecule.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

An embodiment of the disclosure will now be described by example only and with reference to the following figures and examples:

Table 1 and Table 2 illustrates mRNA expression of ASPP1, ASPP2 and iASPP in human breast-tumor samples (DCIS, grade 1-2-3) expressing either wild type and mutant p53. Down arrow, reduced expression; up arrow, overexpression; -, similar expression.

### BRIEF DESCRIPTION OF THE FIGURES

FIGS. 1A and 1B illustrates that the down regulation of ASPP1, ASPP2 or up regulation of iASPP are more frequent in tumours homozygous of p53Pro72 than of p53Arg72.

FIGS. 2A-D illustrate that the ASPP family members selectively regulate the transactivation and apoptotic fimction of a common polymorphism variant of p53, p53Pro72. Saos-2 cells were transfected with two p53 polymorphism variants, p53Pro72 or p53Arg72, in the presence or absence of ASPP1, ASPP2 (FIGS. 2A and 2B) or iASPP (FIGS. 2C and 2D) as indicated. The bar graphs represent the mean value of at least three independent experiments. The expression levels of the p53Pro72, p53Arg72, ASPP1, ASPP2 and iASPP are shown in the lower panels.

FIGS. 3A-B illustrate that the two polymorphism variants, p53Pro72 and p53Arg72, have different abilities to transactivate the promoters of PIG3, Bax but not mdm2 (A) and to induce apoptosis (B) in Saos-2 and H1299 cells.

FIGS. 4A-D illustrate that endogenous iASPP expression level dictates the activities of p53Arg72 and p53Pro72. Western blot shows the expression levels of the ASPP family members in H1299 and Saos-2 cells (A). Expression of ASPP1 and ASPP2 enhanced the apoptotic fimction of p53Pro72 to a similar level as that seen with p53Arg72 in H1299 cells (B). RNAi of iASPP was able to significantly enhance the ability of p53Pro72 but less so of p53Arg72 to transactivate Bax promoter in H1299 and Saos-2 cells. The ability of iASPP RNAi to enhance the transactivation fimction of p53 polymorphic variants on mdm2 promoter is similar in both cell lines (C). Finally, RNAi of iASPP was also able to significantly enhance the apoptotic function of p53Pro72 but not p53Arg72 in H1299 cells (FIG. 4D, left panel). Under the same conditions, much less effects were observed with RNAi of iASPP in Saos-2 cells (FIG. 4D, right panel).

FIGS. 5A and 5B illustrates that the ASPP family of proteins have higher binding affinity to p53Pro72 than p53Arg72. The two p53 polymorphism variants, p53Pro72 and p53Arg72, ASPP1, ASPP2 and iASPP were in vitro translated and labelled with ³⁵S-methionine. Both ASPP1 and ASPP2 were tagged with V5 epitope and they were immunoprecipitated with antibody V5 (IP:V5). The ability of individual ASPP family members to selectively complex with endogenous p53Pro72 but less so with p53Arg72 were detected in a colorectal cell line RKO which expresses p53Pro72 and p53Arg72 at a similar level (FIG. 5B). RKO cells treated with etoposide (10 μM) for 8 hours were labelled as (+). The antibodies used to immunoprecipitate endogenous ASPP1, ASPP2 and iASPP were rabbit polyclonal antibodies 1.88, DX77 and iASPP. 18 respectively and the amounts of ASPP1, ASPP2 and IASPP proteins immunoprecitated down by the antibodies were detected with mouse monoclonal antibodies LX011, DX54.10 and LX049 respectively. The presence of the two p53 polymorphism variants, p53Pro72 and p53Arg72, were detected by the antibody DO.1 and is labelled as p53P or p53R respectively.

FIG. 6 is the nucleic acid sequence of human iASPP.

FIG. 7A is the nucleic acid sequence of human p53.

FIG. 7B is the amino acid sequence of p53 polymorphic polypeptides.

FIG. 8 is the amino acid sequence of human iASPP.

FIG. 9A is the nucleic acid sequence of human ASPP1.

FIG. 9B is the nucleic acid sequence of human ASPP2.

FIG. 10A is the amino acid sequence of human ASPP1.

FIG. 10B is the amino acid sequence of human ASPP2.

### MATERIALS AND METHODS

Cells were grown in DMEM supplemented with 10% FCS. DO-1 is a mouse anti-p53 antibody. The V5 epitope is recognised by the mouse monoclonal antibody V5. CD20Leu is an FITC conjugated monoclonal antibody specific for the cell surface marker CD20 (Becton Dickinson). The mouse and rabbit antibodies to ASPP1 and ASPP2 were described previously (Rabbit anti-ASPP1 antibody pAb ASPP1.88, Rabbit anti-ASPP2 antibody pAb DX77 Rabbit anti-iASPP antibody pAb iASPP.18, mouse monoclonal anti-ASPP1 antibody LX011, mouse monoclonal anti-ASPP2 antibody DX54.10, mouse monoclonal anti-iASPP antibody LX049). The CMV immediate early promoter drove all expression plasmids used in this study. ASPP1 was tagged with the V5 epitope.

Transactivation Assays

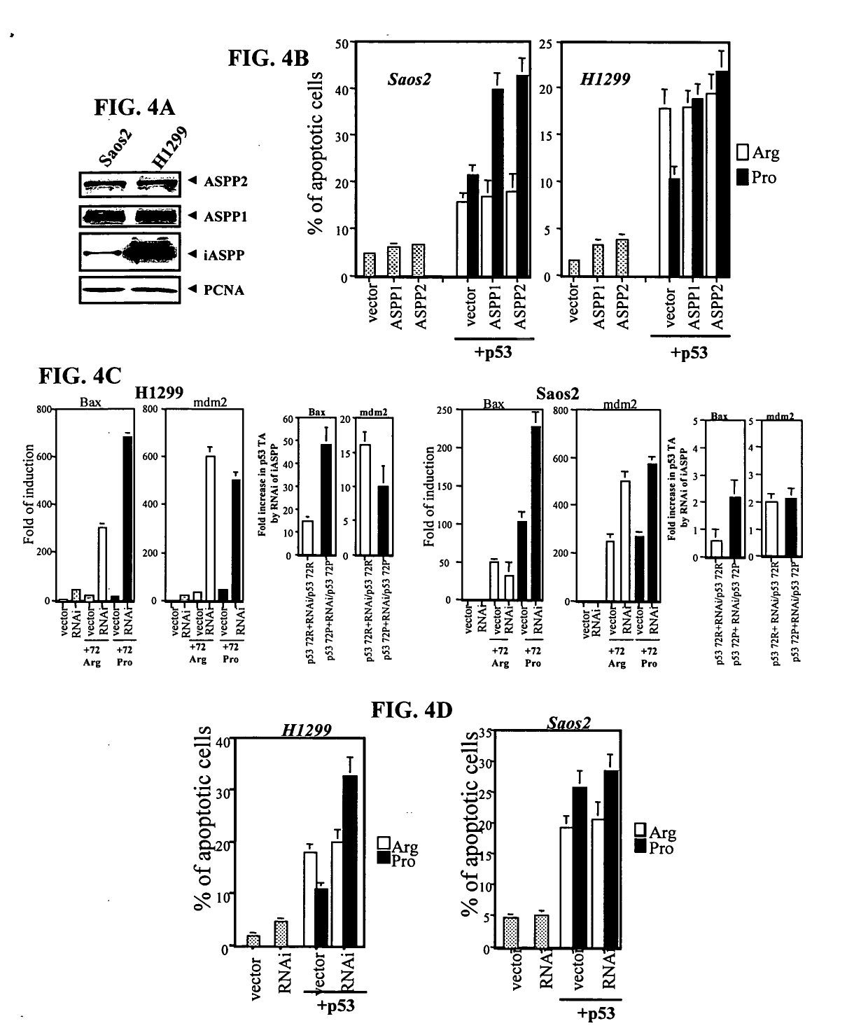

Saos-2 or H1299 cells (5×10⁵) were plated 24 hours prior to transfection in 6 cm dishes. All transactivation assays contained 1 μg of reporter plasmid. 50 ng of p53Pro72 or p53Arg72, 4 μg of ASPP1 or ASPP2, 2 μg of iASPP expression plasmids were used as indicated. In FIG. 4C cells were also co-transfected with 3 μg of pSuper plasmid containing iASPP RNAi as indicated. Cells were lysed in Reporter Lysis Buffer 16-24 hours after transfection and assayed using the Luciferase Assay kit (Promega, Wis., USA). The fold activation of a particular reporter was determined by the activity of the transfected plasmid divided by the activity of vector alone.

Flow Cytometry

Cells (10⁶) were plated 24-48 hours prior to transfection in 10 cm plates. All cells were transfected with 2 μg of a plasmid expressing CD20 as a transfection marker. Transfection consisted of 1 μg of human p53Pro72 or p53Arg72, 10 μg of ASPP1 or ASPP2, 4 μg of iASPP and 8 μg of pSuper plasmid containing RNAi of iASPP as indicated. 36 hours after the transfection, both attached and floating cells were harvested and analysed as described.

Protein Biochemistry

For western blotting, cells were lysed in either NP40 lysis buffer or luciferase reporter lysis buffer. Between 15-100 μg of protein extract was loaded on SDS-PAGE gels. For immunoprecipitation, cells were lysed in NP40 lysis buffer and pre-cleared with protein G beads for 1 hour at 4° C. The protein concentration was determined and then 1-2 mg of the extract was incubated with antibody pre-bound to protein G beads for 4 hours or overnight at 4° C. The beads were washed twice in NP40 lysis buffer and twice in NET buffer. The IP beads were mixed with 5× sample buffer and loaded onto an SDS-PAGE gel. The gels were wet transferred on to Protran nitrocellulose membrane and the resulting blots were first incubated with primary antibody and subsequently with the appropriate secondary HRP conjugated antibody (Dako). The blot was exposed to hyperfilm following the use of ECL substrate solution (Amersham Life Science).

In Vitro Translation and In Vitro Immunoprecipitation

p53Pro72, p53Arg72, ASPP1, ASPP2 and iASPP were in vitro translated and labelled with ³⁵S -Methionine using the TNT T7 Quick coupled Transcription/Translation System (Promega). The lysates containing indicated proteins were incubated at 30° C. for 1 hour. The anti-ASPP1, ASPP2 or iASPP antibodies immobilised on protein G agarose beads were added to the binding reactions and incubated on a rotating wheel at 4° C. for 16 hours. The beads were then washed with PBS. The bound proteins were released in SDS gel sample buffer and analysed by 10% SDS-polyacrylamide gel electrophoresis (PAGE). Results were visualised using autoradiography for ³⁵S -Methionine labelled proteins. For immunoprecipitation using cell lysates, the expression of endogenous ASPP1, ASPP2, iASPP, p53Pro72 and p53Arg72 were detected by western blot using antibodies specific to these proteins derived from different species from that used in IP.

Construction of siRNA of IASPP

Oligonucleotides (19 bp) derived from iASPP were ligated into pSuper expression plasmids as described previously¹¹ . The plasmids containing correct 19 bp oligonucleotides of iASPP were confirmed by sequencing. The sequences of iASPP oligonucleotides used in this study are as follow. The complete insert sequences of iASPP used in this study are (cDNA stretches shown in upper case) as follow:

Sense (S) and Antisense (A) Oligos for iASPP

(SEQ ID NO: 9)

S:5′-gatccccTGTCAACTCCCCCGACAGCttcaagagaGCTGTCGGGG

GAGTTGACAtttttggaaa 3′
(SEQ ID NO: 10)

A:5′agcttttccaaaaaTGTGAAGTCCCCCGACAGCtctcttgaaGCTG

TCGGGGGAGTTGACAggg 3′

Real Time RT-PCR of Tumour and Matched Normal Controls

The breast cancers were all ductal carcinomas of no special type. The presence of an adequate proportion of tumour tissue was confirmed histologically prior to analysis. Codon 72 single nucleotide polymorphism was performed as described previously¹². Mutations in p53 were analysed by single strand conformational polymorphism (SSCP) and sequencing as described. The expression of the ASPP family members was performed using TaqMan PCR.

The primer sequences are as follows.

| | |
|---|---|
| ASPP 1 | |
| forward: ccagcaagccacaccacctaagaattac | (SEQ ID NO: 11) |
| reverse: tgaacccgaaggtaaaacgggcttac | (SEQ ID NO: 12) |
| probe: [FAM]-ccggcagcacacagcgccttaa-[TAMRA] | (SEQ ID NO: 17) |
| ASPP2 | |
| forward: gaagactcggtgagcatgcg | (SEQ ID NO: 13) |
| reverse: gcgatacgctctgagccagt | (SEQ ID NO: 14) |
| probe:[FAM]-ccgcctgaaatcaccggggcaggtct-[TAMRA] | (SEQ ID NO: 18) |
| iASPP | |
| forward:caggcggtgaaggagatgaacg | (SEQ ID NO: 15) |
| reverse: aaatccacgatagagtagttggcgc | (SEQ ID NO: 16) |
| probe: [FAM]-cccgagccagcccaacgagg-[TAMRA] | (SEQ ID NO: 19) |

### EXAMPLES

The most common polymorphism, encoded by a single nucleotide polymorphism (SNP), in p53 is located at residue 72 and the natural occurring amino acid at this residue is either Proline or Arginine. Interestingly, codon 72 of p53 is located within the proline rich region of p53, a region known to be required for p53 to induce apoptosis but not cell cycle arrest. The proline rich region of p53 is also required for p53 to transactivate its target genes such as PIG3 but not p21waf1 or mdm2¹,². Interestingly the binding of ASPP1 and ASPP2 to p53 specifically enhances the ability of p53 to bind and transactivate pro-apoptotic genes such as Bax, PIG3 but not p21 waf1 or mdm2³. The identification of the ASPP family of proteins as specific regulators of p53 revealed a novel mechanism by which the apoptotic function of p53 is regulated. The apoptotic function of p53 is stimulated by ASPP1, ASPP2 and inhibited by iASPP. The regulation of p53 by the ASPP family members is evolutionarily conserved, from worm to human^{3,4}. Moreover, deregulated expression of the ASPP family members (reduced expression of ASPP1 and ASPP2 and increased expression of iASPP) was also observed in a large percentage of breast tumours expressing wild type p53 ⁴. The ASPP family of proteins contain SH3 domain. Proteins with these domains have high affinity for the proline rich sequence, raising the possibility that the two-polymorphism variants of p53 may be subject to different regulation by the ASPP family of proteins.

### Example 1

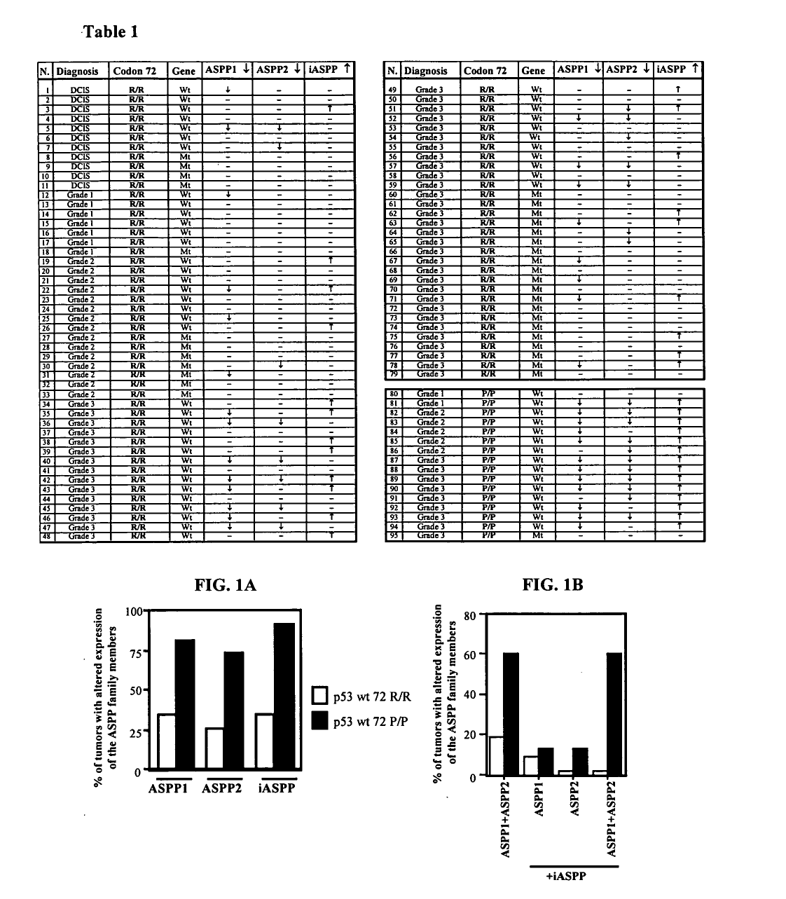

To address this issue, we examined expression of the ASPP family members in a panel of 95 human breast carcinomas homozygous for p53Arg72 (n=62) or p53Pro72 (n=16)(table 1) using real time RT-PCR. Consistent with our previous study^{3,4} abnormal expression of the ASPP family members was detected in around 24% of the tumours expressing mutant p53 but in around 50% of the tumours expressing wild type p53 (table 1 and table 2). Detailed analysis revealed that the frequency of abnormal expression of the ASPP family members is higher in a panel of wild type p53 expressing human breast tumours homozygous for p53Pro72 than those with p53Arg72 (table 2). Among the 47 wild type p53 expressing tumours homozygous for p53Arg72, reduced expression of ASPP1 or ASPP2 (50% less than that detected in the matched normal controls) were observed in 34% (16/47) and 26% (12/47) of the tumours. Interestingly, down regulation of ASPP1 or ASPP2 expression was observed in 80% (12/15) or 73% (11/15) of wild type p53 expressing tumours homozygous for p53Pro72. Similarly, while iASPP was over expressed in 90% of the tumours homozygous for p53Pro72 (14/15), only 32% (15/47) of the tumours homozygous for p53Arg72 over expressed iASPP (table 1, table 2 and FIG. 1A). Deregulated expression of two or more ASPP family members within the same tumour tends to occur in wild type p53 expressing tumours and the tumours homozygous for p53Pro72. In particular, reduced expression of ASPP1+ASPP2 or reduced expression of ASPP1+ASPP2 together with increased expression of iASPP were only seen in wild type p53 expressing tumours. These altered expression patterns of the ASPP family members occurred in 60% of the tumours homozygous for p53Pro72. This is in contrast to that seen in tumours homozygous for p53Arg72 where only 19% of them showed reduced expression of ASPP1+ASPP2 and 2% of them have reduced expression of ASPP1+ASPP2 and increase expression of iASPP (table 2, FIG. 1B). Together, these results suggest that selective pressure to alter the expression of the ASPP family members in tumours homozygous for p53Pro72 is much higher than those with p53Arg72.

TABLE 2
Alteration in ASPP expression

| | wild type p53 | | | mutant p53 | | |
|---|---|---|---|---|---|---|
| | RR | PP | total | RR | PP | total |
| | (n = 47) | (n = 15) | (n = 62) | (n = 32) | (n = 1) | (n = 33 |
| ASPP1 | 16 (34%) | 12 (80%) | 29 (47%) | 8 (25%) | 0 | 8 (24%) |
| ASPP2 | 12 (26%) | 11 (73%) | 25 (40%) | 3 (9%) | 0 | 3 (9%) |
| iASPP | 15 (32%) | 14 (93%) | 30 (48%) | 6 (19%) | 0 | 6 (18%) |
| ASPP1 + ASPP2 | 9 (19%) | 9 (60%) | 19 (31%) | 0 | 0 | 0 |
| ASPP1 + iASPP | 4 (9%) | 2 (13%) | 6 (10%) | 3 (9%) | 0 | 3 (9%) |
| ASPP2 + iASPP | 1 (2%) | 2 (13%) | 3 (5%) | 0 | 0 | 0 |
| ASPP1 + ASPP2 + iASPP | 1 (2%) | 9 (60%) | 11 (18%) | 0 | 0 | 0 |

### Example 2

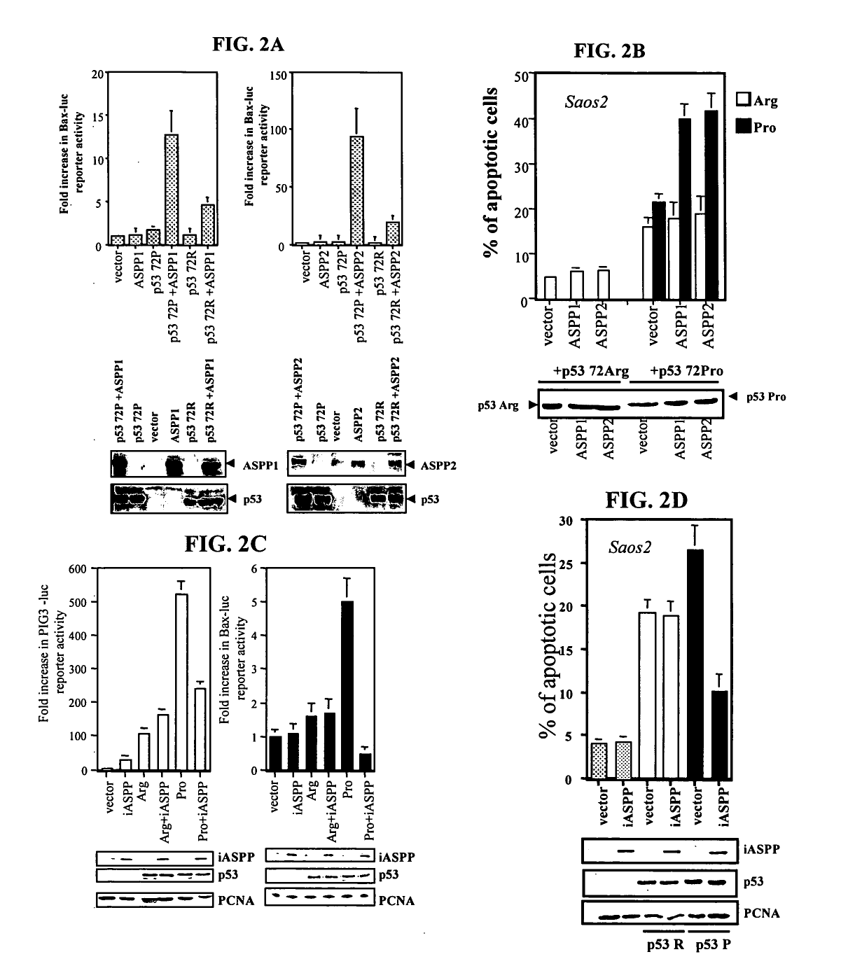

To understand the mechanisms underlying this bias, we investigated whether the p53 polymorphic variants are subject to differential regulation by the ASPP family members. In Saos-2 cells, the transcriptional activity of p53Pro72 on the promoters of Bax and PIG3 is similar to or slightly greater than p53Arg72 (FIG. 2). Expression of ASPP1 and ASPP2, however, significantly enhanced the transcriptional activity of p53Pro72 on Bax promoter, whereas ASPP1 and ASPP2 only had very little effect on the transactivation function of p53Arg72, despite expressing similar levels of ASPP1, ASPP2 and p53 (FIG. 2A). The ability of ASPP1 and ASPP2 to selectively enhance the apoptotic function of p53Pro72 but not p53Arg72 was also observed in Saos-2 cells (FIG. 2B). Moreover, iASPP also selectively inhibited the transactivation and apoptotic function of p53Pro72 but not p53Arg72 (FIGS. 2C and 2D).

### Example 3

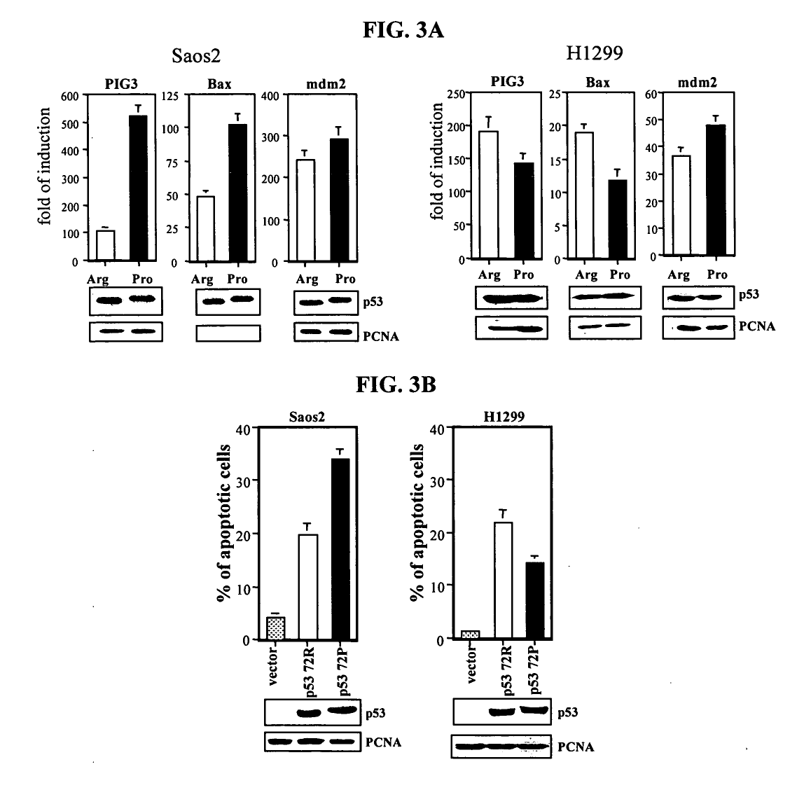

In the absence of any exogenous ASPP expression, however, the activities of p53Pro72 and p53Arg72 differ in different cells. In Saos-2 cells, p53Pro72 was more active than p53Arg72 to transactivate the Bax and PIG3 promoters (FIG. 3A, left panel). However opposite results were obtained in H1299 cells (FIG. 3A, right panel). The ability of the two polymorphic p53 variants to transactivate the mdm2 promoter was comparable in both H1299 and Saos-2 cells (FIG. 3A). Similarly, p53Pro72 was slightly more active than p53Arg72 to induce apoptosis in Saos-2 cells but opposite results were obtained in HI 299 cells (FIG. 3B). The difference seen here was not caused by a difference in the expression levels of p53Pro72 or p53Arg72, similar amounts of p53 were detected in both cell lines. Since the ASPP family members selectively regulate the activities of p53Pro72, perhaps the endogenous expression levels of the ASPP family members might be responsible for the differences in the activities of the two polymorphic p53 variants. The expression levels of ASPP1, ASPP2 and iASPP in H1299 and Saos-2 cells were measured.

### Example 4

Although the expression levels of ASPP1 and ASPP2 were similar in both cell lines, the expression level of iASPP was 3-5 fold higher in H1299 cells than in Saos-2 cells (FIG. 4A). Hence it was possible that the endogenous iASPP level might dictate the activities of the two polymorphic p53 variants. The inhibitory activities of endogenous iASPP were counteracted by the introduction of exogenous ASPP1 or ASPP2. As a result, exogenous ASPP1 and ASPP2 selectively enhanced the transactivation and apoptotic function of p53Pro72 but not p53Arg72 in Saos-2 and H1299 cells (FIG. 4B). Moreover, the added expression of ASPP1 and ASPP2 was able to enhance the activities of p53Pro72 to level similar to that seen with p53Arg72 in H1299 cells (FIG. 4B, right panel). The ability of iASPP to influence the activities of p53Pro72 and p53Arg72 was further tested using RNA interference to reduce the expression of endogenous iASPP. In Saos-2 cells, the expression of iASPP RNAi stimulated the transactivation function of p53Pro72 on the Bax promoter, but had very little effect on p53Arg72 (FIG. 4C). The effect of iASPP RNAi on the transactivation function of p53 polymorphism variants on mdm2 promoter was very similar (FIG. 4C). Similar results were also obtained in H1299 cells. The expression of iASPP RNAi increased the transactivation function of p53Arg72 and p53Pro72 on the promoters of Bax and mdm2. However, the fold increase in p53Pro72 activity on the Bax promoter was much greater than that of p53Arg72 (FIG. 4C). Additionally, RNAi of iASPP also enhanced the apoptotic function of p53Pro72 in both H1299 and Saos-2 cells. The extent of increase in the activities of p53Pro72 in H1299 cells was much greater than that seen in Saos-2 cells (FIGS. 4C and 4D) consistent with the observation that the expression levels of iASPP is higher in H1299 cells than in Saos-2 cells. These results demonstrate that the ASPP family members, iASPP in particular, determine the apoptotic function of the two polymorphic variants of p53.

### Example 5

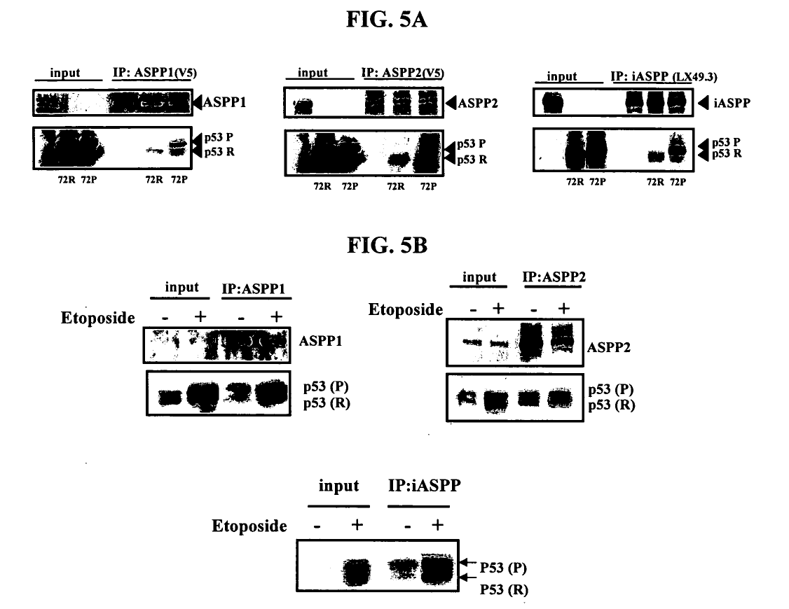

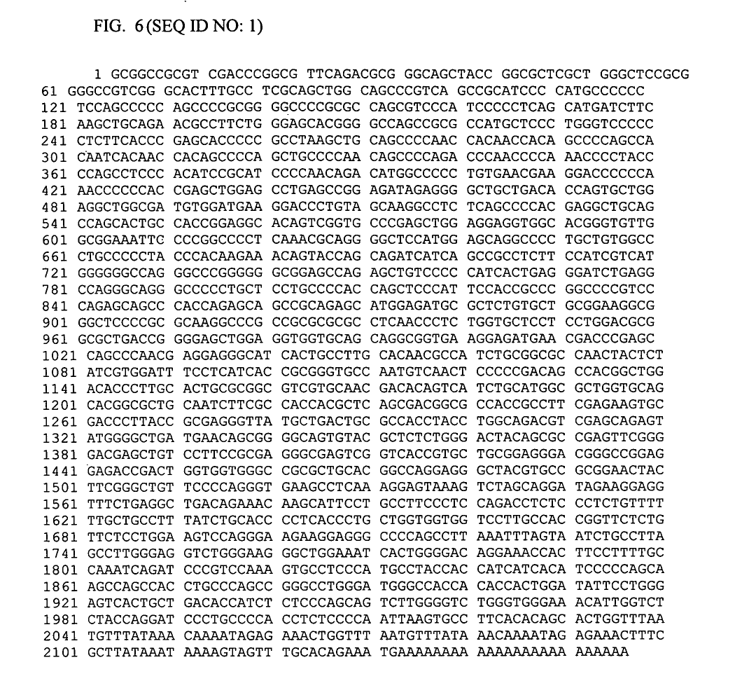

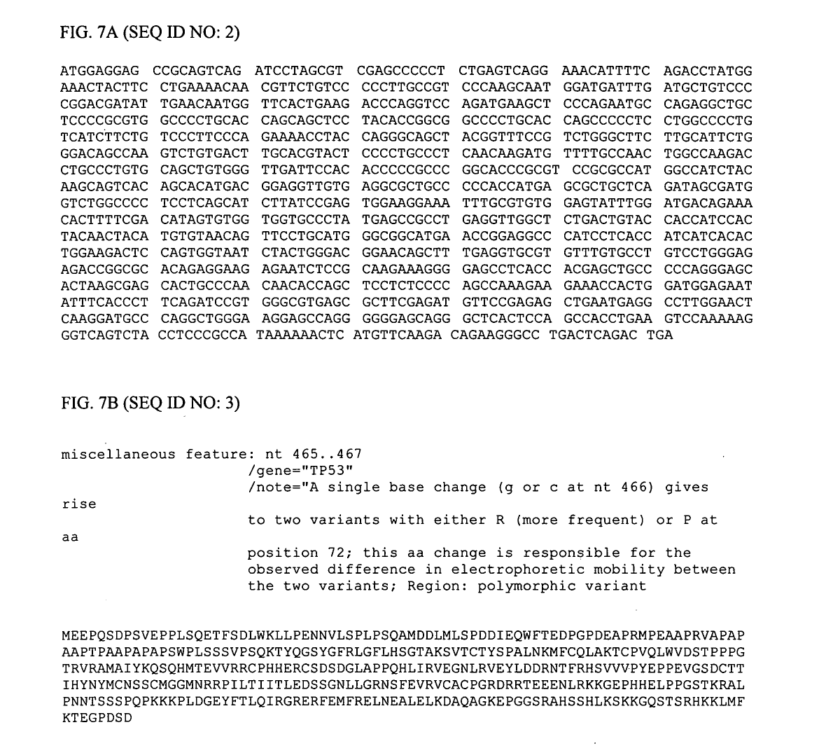

Although the major binding site of ASPP on p53 is in the DNA binding region of p53, the proline rich region of p53 may influence the interaction between p53 and ASPP, since the ASPP family of proteins contain SH3 domain which is known to bind proline-rich region of a protein⁵. This was initially tested using in vitro translated p53Arg72, p53Pro72, ASPP1, ASPP2 and iASPP. As shown in FIG. 5A, all the ASPP family members bound p53Pro72 better than p53Arg72. The preferential binding between p53Pro72 and the ASPP family members was also investigated in RKO cells, a colorectal cell line expressing wild type p53 and heterozygous for p53Pro72/p53Arg72. As shown in FIG. 5B, the amount of p53Pro72 co-immunoprecipitated with iASPP was clearly larger than that of p53Arg72, even though similar amount of p53Pro72 and p53Arg72 were expressed in RKO cells (FIG. 5B). Selective binding of ASPP2 to p53Pro72 was also seen. A similar pattern of selectivity towards p53Pro72 was also seen with ASPP1 but to a lesser extent. These data demonstrate that iASPP has the strongest selectivity towards p53Pro72.

The findings reported here provide the first molecular explanation why the two polymorphic p53 variants have significantly different biological activity. We demonstrated recently that the regulation of p53 by the ASPP family members is evolutionarily conserved. We have also shown that iASPP is the most conserved member of the ASPP family and the only ASPP family member present in C.elegans⁴. Hence it is interesting that the ASPP family members selectively regulate a more ancient polymorphic p53 variant, p53Pro72. The polymorphism of p53 at codon 72 only exists in human and p53Arg72 is human specific. Moreover, the frequency of allele encoding p53Pro72 varies among different ethnic populations. The number of individuals homozygous for p53Pro72 is closely linked to the latitude and it is much higher in the blacks living in the equator, suggesting that p53Pro72 is selected for by the environment with high levels of UV light^{6,7}. Molecular explanation of such selection may due to the fact that the ASPP family of proteins selectively regulate the apoptotic function of p53Pro72. In response to various stress signals, two different pathways are involved in regulating the apoptotic function of p53Pro72 or p53Arg72. Being able to escape the negative regulation of iASPP may be one of the reasons why p53Arg72 is evolved in human. It also explains why it could more active than p53Pro72 to induce apoptosis⁸. The most efficient way to inactivate the apoptotic fluction of p53Arg72 is mutation. Consistent with this, the percentage of mutant p53 expressing tumours homozygous for p53Arg72 is much higher than those with p53Pro72 (42% and 6% respectively) in the panel of breast tumours examined. Being a more potent inhibitor of p73, there is also a selective advantage to mutate p53Arg72 in tumours⁹. In contrast, either reducing the expression of ASPP1, ASPP2 or increasing the expression of iASPP in addition to mutating p53 itself can achieve inactivation of p53Pro72. Therefore the ASPP family of proteins provided another level of regulation of p53Pro72. As a result, the integrity of p53Pro72 is better protected than p53Arg72 in normal cells in response to signals that induce the apoptotic function of p53. This may be why the percentage of p53Pro72 homozygous carrier is the highest in the ethnic populations living in the environment consistently exposed to high dose of p53 inducing agents such as UV. Nevertheless, homozygosity for p53pro72 does not necessarily mean protection against p53 mutation in other types of cancer as the expression levels of the ASPP family members vary dramatically among different tissues (data not shown)¹⁰. Only when the expression levels of the ASPP family members are taken into consideration, can we draw clear conclusions with regard to whether, when and where the expression of certain polymorphic p53 variants is associated with cancer susceptibility. The results shown here will also guide us to develop better strategies to treat cancer according to their p53 polymorphism and ASPP expression patterns.

References

1. Walker & Levine Identification of the novel p53 functional domain which is necessary for efficient growth suppression. Proc. Natl. Acad. Sci. USA 93, 15335-40 (1996).
2. Venot et al. The requirement for the p53 proline-rich functional domain for mediation of apoptosis is correlated with specific PIG3 gene transactivation and with transcriptional repression. EMBO 17, 4668-79 (1998).
3. Samuels-Lev et al. ASPP proteins specifically stimulate the apoptotic function of p53. Mol. Cell 8, 781-94 (2001).
4. Bergamaschi et al. iASPP oncoprotein is a key inhibitor of p53 conserved from worm to human. Nature Genetics 33, 162-7 (2003).
5. Gorina & Pavletich Structure of the p53 tumor suppressor bound to the ankyrin and SH3 domains of 53BP2. Science 274, 1001-5 (1996).
6. Sjalander et al. p53 polymorphisms and haplotypes in different ethnic groups. Hum Hered 45, 144-9 (1995).
7. Beckman et al. Is p53 polymorphism maintained by natural selection? Hum Hered 44, 266-70 (1994).
8. Dumont et al. The codon 72 polymorphic variants of p53 have markedly different apoptotic potential. Nat Genet 33, 357-65 (2003).
9. Marin, M. C. et al. A common polymorphism acts as an intragenic modifier of mutant p53 behaviour. Nat Genet 25, 47-54 (2000).
10. Iwabuchi et al. Two cellular proteins that bind to wild-type but not mutant p53. Proc.
   Nati. Acad. Sci. USA 91, 6098-102 (1994).
11. Brummelkamp et al. A system for stable expression of short interfering RNAs in mammalian cells. Science 296, 550-3 (2002).
12. Bergamaschi et al. p53 polymorphism influences response in cancer chemotherapy via modulation of p73-dependent apoptosis. Cancer Cell 3, 387-402 (2003).

1. A screening method for the identification of agents which modulate the interaction of a polypeptide encoded by a nucleic acid molecule selected from the group consisting of:
a) a polypeptide encoded by a nucleic acid molecule comprising a nucleic acid sequence as represented in SEQ ID NO: 1;
b) a polypeptide encoded by a nucleic acid molecule which hybridises to the nucleic acid molecule in (a) and which inhibits the apoptotic activity of p53;
c) a polypeptide encoded by a nucleic acid molecule consisting of a nucleic acid sequence which is degenerate as a result of the genetic code to a nucleic acid molecule as defined in (a) and (b);
with a p53 polymorphic polypeptide variant wherein said variant is modified by substitution of an amino acid residue encoded by codon 72 of the nucleic acid sequence as represented in SEQ ID NO: 2 comprising:
i) forming a preparation comprising said polypeptide and said p53 variant;
ii) adding at least one candidate agent to be tested; and
iii) determining the effect, or not, of said agent on the interaction of said polypeptide with said p53 variant.

2. The of claim 1, wherein said polypeptide is encoded by a nucleic acid molecule consisting of a nucleic acid sequence as represented by SEQ ID NO: 1.

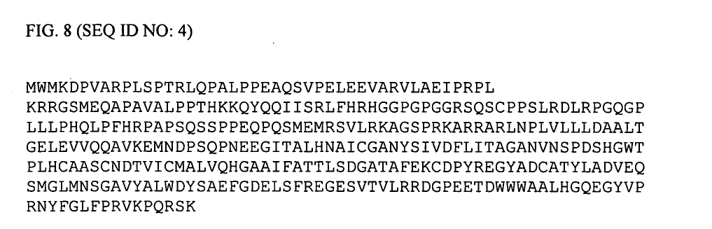

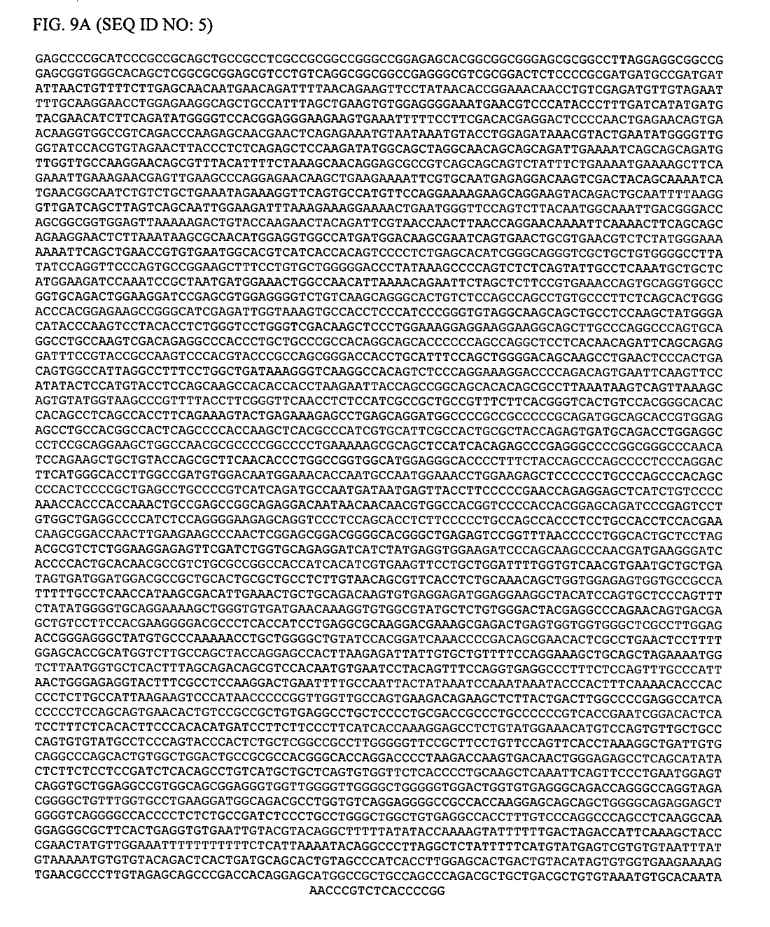

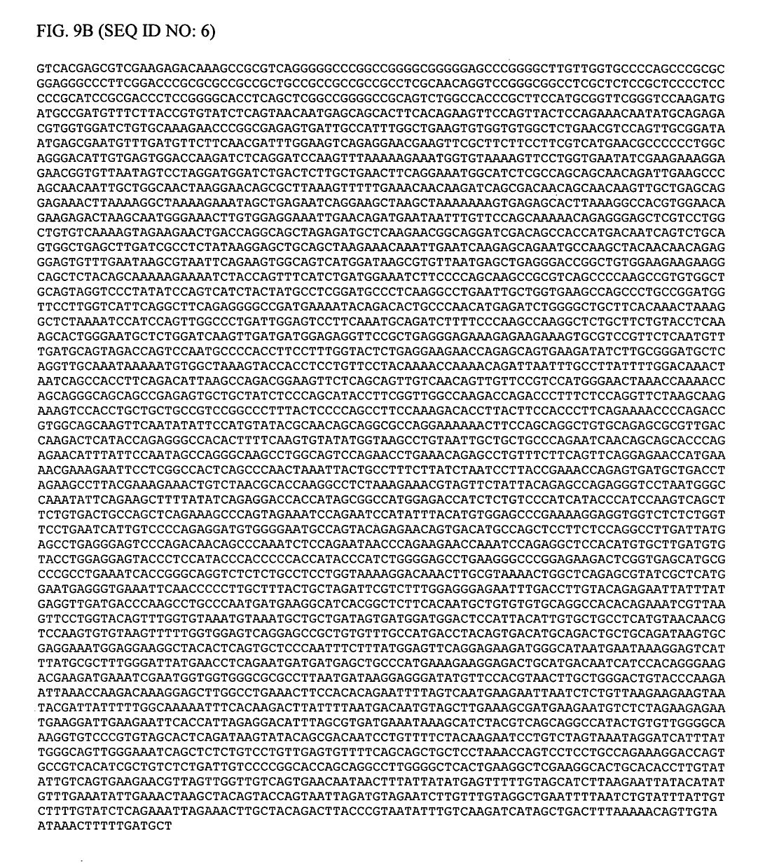

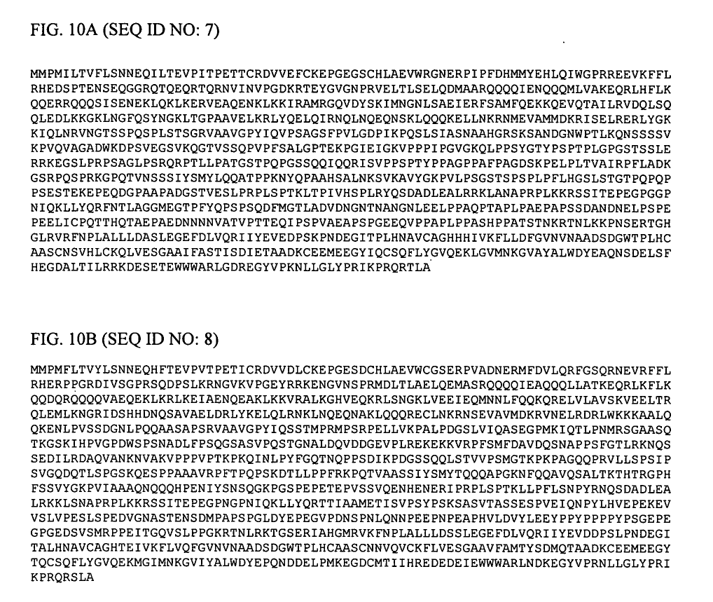

3. The method of claim 1, wherein said polypeptide is represented by the amino acid sequence as shown in SEQ ID NO: 4, or a variant polypeptide wherein said variant polypeptide is modified by addition, deletion or substitution of at least one amino acid residue which varies with respect to the amino acid sequence shown in FIG. 8.

4. The method of claim 1, wherein said p53 variant varies at codon 72 wherein said codon encodes a proline amino acid residue.

5. The method of claim 1, wherein said agent an antagonist.

6. The method of claim 1, wherein said agent is a polypeptide.

7. The method of claim 6, wherein said polypeptide is an antibody or active binding part thereof.

8. The method of claim 7, wherein said antibody or binding part is a monoclonal antibody.

9. The method of claim 7, wherein said antibody part is a single chain antibody variable region fragment or a domain antibody fragment.

10. The method of claim 7, wherein said antibody is a humanised antibody.

11. The method of claim 7, wherein said antibody is a chimeric antibody.

12. The method of claim 1, wherein said agent is a peptide.

13. The method of claim 12, wherein said peptide is a modified peptide.

14. The method of claim 1, wherein said agent is an aptamer.

15. The method of claim 1, wherein said method further comprises testing the candidate agent for activity with respect to a second different p53 polymorphic polypeptide variant.

16. The method of claim 15, wherein said p53 variant is modified by substitution of an amino acid residue encoded by codon 72 of the nucleic acid sequence as represented in SEQ ID NO: 3.

17. The method of claim 16, wherein said p53 variant varies at codon 72 wherein said codon encodes an arginine amino acid residue.

18. The method of claim 1, wherein said preparation comprises a cell transfected with a first nucleic acid molecule selected from the group consisting of:
i) a nucleic acid molecule comprising a nucleic acid sequence as represented in SEQ ID NO: 1;
ii) a nucleic acid molecule which hybridises to the nucleic acid molecule in (i) and which encodes a polypeptide which inhibits the apoptotic activity of p53;
iii) a nucleic acid molecule consisting of a nucleic acid sequence which is degenerate as a result of the genetic code to a nucleic acid molecule as defined in (i) and (ii); and a second nucleic acid molecule which encodes a p53 polymorphic polypeptide variant wherein said variant is modified by substitution of an amino acid residue encoded by codon 72 of the nucleic acid sequence as represented in SEQ ID NO: 2.

19. The method of claim 18, wherein said p53 variant varies at codon 72 wherein said codon encodes a proline amino acid residue.

20. A cell transfected with a first nucleic acid molecule selected from the group consisting of:
i) a nucleic acid molecule comprising a nucleic acid sequence as represented in SEQ ID NO: 1;
ii) a nucleic acid molecule which hybridises to the nucleic acid molecule in (i) and which encodes a polypeptide which inhibits the apoptotic activity of p53;
iii) a nucleic acid molecule consisting of a nucleic acid sequence which is degenerate as a result of the genetic code to a nucleic acid molecule as defined in (i) and (ii); and a second nucleic acid molecule which encodes a p53 polymorphic polypeptide variant wherein said variant is modified by substitution of an amino acid residue encoded by codon 72 of the nucleic acid sequence as represented in SEQ ID NO: 2.

21. The cell of claim 20, wherein said cell is transfected with a second nucleic acid molecule which encodes a p53 variant which varies at codon 72 wherein said codon encodes a proline amino acid residue.

22. The cell of claim 20, wherein said cell is a cancer cell.

23. A screening method for the identification of agents which modulate the interaction of a polypeptide encoded by a nucleic acid molecule selected from the group consisting of:
a) a polypeptide encoded by a nucleic acid molecule comprising a nucleic acid sequence as represented in SEQ ID NO: 5 or 6;
b) a polypeptide encoded by a nucleic acid molecule which hybridises to the nucleic acid molecule in (a) and which stimulates the apoptotic activity of p53;
c) a polypeptide encoded by a nucleic acid molecule consisting of a nucleic acid sequence which is degenerate as a result of the genetic code to a nucleic acid molecule as defined in (a) and (b);
with a p53 polymorphic polypeptide variant wherein said variant is modified by substitution of an amino acid residue encoded by codon 72 of the nucleic acid sequence as represented in SEQ ID NO: 2 comprising:
i) forming a preparation comprising said polypeptide and said p53 variant;
ii) adding at least one candidate agent to be tested; and
iii) determining the effect, or not, of said agent on the interaction of said polypeptide with said p53 variant.

24. The method of claim 23, wherein said polypeptide is encoded by a nucleic acid molecule consisting of a nucleic acid sequence as represented by SEQ ID NO: 5 or 6.

25. The method of claim 23, wherein said polypeptide is represented by the amino acid sequence as shown in SEQ ID NO: 7 or 8, or a variant polypeptide wherein said variant polypeptide is modified by addition, deletion or substitution of at least one amino acid residue which varies with respect to the amino acid sequence shown in SEQ ID NO: 7 or 8.

26. The method of claim 20, wherein said agent is an agonist.

27. The method of claim 20, wherein said agent is a polypeptide, antibody, antibody fragment, peptide or aptamer.

28. The method of claim 20, wherein said preparation comprises a cell transfected with a first nucleic acid molecule selected from the group consisting of:
i) a nucleic acid molecule comprising a nucleic acid sequence as represented in SEQ ID NO: 5 or 6;
ii) a nucleic acid molecule which hybridises to the nucleic acid molecule in (i) and which encodes a polypeptide which stimulates the apoptotic activity of p53;
iii) a nucleic acid molecule consisting of a nucleic acid sequence which is degenerate as a result of the genetic code to a nucleic acid molecule as defined in (i) and (ii); and a second nucleic acid molecule which encodes a p53 polymorphic polypeptide variant wherein said variant is modified by substitution of an amino acid residue encoded by codon 72 of the nucleic acid sequence as represented in SEQ ID NO: 2.

29. The method according to claim 28 wherein said p53 variant varies at codon 72 wherein said codon encodes a proline amino acid residue.

30. A cell transfected with a first nucleic acid molecule selected from the group consisting of:
i) a nucleic acid molecule comprising a nucleic acid sequence as represented in SEQ ID NO: 5 or 6;
ii) a nucleic acid molecule which hybridises to the nucleic acid molecule in (i) and which encodes a polypeptide which stimulates the apoptotic activity of p53;
iii) a nucleic acid molecule consisting of a nucleic acid sequence which is degenerate as a result of the genetic code to a nucleic acid molecule as defined in (i) and (ii); and a second nucleic acid molecule which encodes a p53 polymorphic polypeptide variant wherein said variant is modified by substitution of an amino acid residue encoded by codon 72 of the nucleic acid sequence as represented in SEQ ID NO: 2.

31. The cell of claim 30, wherein said cell is transfected with a second nucleic acid molecule which encodes a p53 variant which varies at codon 72 wherein said codon encodes a proline amino acid residue.

32. The cell of claim 30, wherein said cell is a cancer cell.

33. A method of diagnosing an animal, comprising:
providing a cell or tissue sample to be tested;
determining the p53 genotype of said animal from analysis of said sample; and optionally
determining the expression of at least one gene product encoded by the genes ASPP1, ASPP2 or iASPP.

34. The method of claim 33, wherein said p53 genotype is p53pro72 or p53arg72.

35. The method of claim 34, wherein said method of diagnosis further includes a determining a treatment regime for said animal by the combination of p53 genotype and the expression status of at least one gene product encoded by ASPP1, ASPP2 or iASPP.

36. The method of claim 33, wherein the animal is a human.

37. The method of claim 33, wherein said cell or tissue sample is a breast sample.

38. A composition comprising at least one nucleic acid molecule wherein said nucleic acid molecule encodes a p53 polypeptide, or sequence variant thereof, and at least one member of the ASPP family of pro-apoptotic polypeptides.

39. A composition comprising a first nucleic acid molecule selected from the group consisting of:
i) a nucleic acid molecule comprising a nucleic acid sequence as represented in SEQ ID NO: 5 or 6;
ii) a nucleic acid molecule which hybridises to the nucleic acid molecule in (i) and which encodes a polypeptide which stimulates the apoptotic activity of p53;
iii) a nucleic acid molecule consisting of a nucleic acid sequence which is degenerate as a result of the genetic code to a nucleic acid molecule as defined in (i) and (ii); and a second nucleic acid molecule comprising a nucleic acid sequence selected from the group consisting of:
iv) a nucleic acid molecule consisting of a nucleic acid sequence as represented in SEQ ID NO: 2;
v) a nucleic acid molecule consisting of a nucleic acid sequence which hybridises to the nucleic acid molecule in (iv) and which encodes a polypeptide with the specific activity associated with p53;
vi) a nucleic acid molecule consisting of a nucleic acid sequence which is degenerate as a result of the genetic code to a nucleic acid molecule as defined in (iv) and (v); wherein said nucleic acid molecules are operably linked to a promoter sequence which facilitates the expression of said first and second nucleic acid molecules.

40. The composition of claim 39, wherein said p53 polypeptide is p53pro72.

41. The composition of claim 39, wherein said nucleic acid molecules are part of an expression vector.

42. The composition of claim 39, wherein said composition is a pharmaceutical composition.

43. The composition of claim 42, wherein said composition further comprises at least one further therapeutic agent.

44. The composition of claim 43, wherein said therapeutic agent is a chemotherapeutic agent.

45. The composition of claim 43, wherein said agent is cisplatin; carboplatin; cyclosphosphamide; melphalan; carmusline; methotrexate; 5-fluorouracil; cytarabine; mercaptopurine; daunorubicin; doxorubicin; epirubicin; vinblastine; vincristine; dactinomycin; mitomycin C; taxol; L-asparaginase; G-CSF; etoposide; colchicine; derferoxamine mesylate; or camptothecin.

46. A method of treatment of an animal suffering from a condition which would benefit from the stimulation of apoptosis comprising administering at least one nucleic acid molecule comprising a nucleic acid sequence which encodes a p53 polypeptide, or variant thereof, and a polypeptide which stimulates the proapoptotic activity of said p53 polypeptide wherein said polypeptide is encoded by a nucleic acid molecule comprising a nucleic acid sequence as shown in SEQ ID NO: 5 or 6, or a nucleic acid molecule which hybridises to these sequences.

47. The method of claim 45, wherein said variant p53 polypeptide is p53arg72.

48. The method of claim 45, wherein said variant p53 polypeptide is p53pro72.

49. A method for the diagnosis and treatment of an animal suffering from a condition which would benefit from the stimulation of apoptosis comprising:
providing a cell or tissue sample to be tested;
determining the p53 genotype of said animal from analysis of said sample; and
administering a composition according to claim 38.

50. The method of claim 49, wherein said p53 genotype is p53arg72.

51. The method of claim 49, wherein said p53 genotype is p53pro72.

52. The method of claim 49, wherein said composition comprises at least one nucleic acid molecule comprising a nucleic acid sequence encoding a p53pro72 polypeptide and a nucleic acid sequence encoding a polypeptide which stimulates the proapoptotic activity of said p53 polypeptide wherein said polypeptide is represented by the nucleic acid sequence as shown in SEQ ID NO: 5 or 6 or a nucleic molecule which hybridises to these sequences under high stringency conditions.

53. The method of claim 49, wherein said condition is cancer.

54. A composition comprising at least one nucleic acid molecule comprising a nucleic acid sequence which encodes a p53 polypeptide, or variant thereof, and a nucleic acid molecule which encodes an agent which antagonises the activity of a polypeptide encoded by a nucleic acid molecule comprising a nucleic acid sequence as represented by SEQ ID NO: 1, or a nucleic acid molecule which hybridises to SEQ ID NO: 1 under high stringency conditions.

55. The composition of claim 54, wherein said p53 variant is p53arg72.

56. The composition of claim 54, wherein said p53 variant is p53pro72.

57. The composition of claim 54, wherein said agent is an antisense RNA.

58. The composition of claim 54, wherein said agent is an RNAi molecule.

59. The composition of claim 54, wherein said agent is an antibody, or active binding fragment thereof, which binds the polypeptide as represented by the amino acid sequence shown in SEQ ID NO: 4.

60. The composition of claim 54, wherein said composition comprises at least one further therapeutic agent.

61. The composition of claim 60, wherein said agent is a chemotherapeutic agent.

62. The composition of claim 60, wherein said agent is cisplatin; carboplatin; cyclosphosphamide; melphalan; carmusline; methotrexate; 5-fluorouracil; cytarabine; mercaptopurine; daunorubicin; doxorubicin; epirubicin; vinblastine; vincristine; dactinomycin; mitomycin C; taxol; L-asparaginase; G-CSF; etoposide; colchicine; derferoxamine mesylate; or camptothecin.

63. A method for the treatment of an animal which would benefit from a stimulation of apoptosis comprising administering the composition of claim 54.

64. A method for the diagnosis and treatment of an animal, comprising:
providing a cell or tissue sample to be tested;
determining the p53 genotype of said animal from analysis of said sample; and
administering the composition of claim 54.

65. The method of claim 64, wherein said p53 genotype is p53arg72.

66. The method of claim 64, wherein said p53 genotype is p53pro72.

67. The method of claim 64, wherein said treatment is the treatment of cancer.


SEQUENCE LISTING (19 sequences)

SEQ ID NO: 1 - DNA, 2156 bp, Homo sapiens
gcggccgcgtcgacccggcgttcagacgcgggcagctaccggcgctcgctgggctccgcggggccgtcgggcactttgcctcgcagctggcagcccgtcagccgcatccccatgcccccctccagcccccagccccgcggggccccgcgccagcgtcccatccccctcagcatgatcttcaagctgcagaacgccttctgggagcacggggccagccgcgccatgctccctgggtcccccctcttcacccgagcacccccgcctaagctgcagccccaaccacaaccacagccccagccacaatcacaaccacagccccagctgccccaacagccccagacccaaccccaaacccctaccccagcctcccacatccgcatccccaacagacatggccccctgtgaacgaaggaccccccaaaccccccaccgagctggagcctgagccggagatagaggggctgctgacaccagtgctggaggctggcgatgtggatgaaggaccctgtagcaaggcctctcagccccacgaggctgcagccagcactgccaccggaggcacagtcggtgcccgagctggaggaggtggcacgggtgttggcggaaattccccggcccctcaaacgcaggggctccatggagcaggcccctgctgtggccctgccccctacccacaagaaacagtaccagcagatcatcagccgcctcttccatcgtcatggggggccagggcccggggggcggagccagagctgtcccccatcactgagggatctgaggccagggcagggccccctgctcctgccccaccagctcccattccaccgcccggccccgtcccagagcagcccaccagagcagccgcagagcatggagatgcgctctgtgctgcggaaggcgggctccccgcgcaaggcccgccgcgcgcgcctcaaccctctggtgctcctcctggacgcggcgctgaccggggagctggaggtggtgcagcaggcggtgaaggagatgaacgacccgagccagcccaacgaggagggcatcactgccttgcacaacgccatctgcggcgccaactactctatcgtggatttcctcatcaccgcgggtgccaatgtcaactcccccgacagccacggctggacacccttgcactgcgcggcgtcgtgcaacgacacagtcatctgcatggcgctggtgcagcacggcgctgcaatcttcgccaccacgctcagcgacggcgccaccgccttcgagaagtgcgacccttaccgcgagggttatgctgactgcgccacctacctggcagacgtcgagcagagtatggggctgatgaacagcggggcagtgtacgctctctgggactacagcgccgagttcggggacgagctgtccttccgcgagggcgagtcggtcaccgtgctgcggagggacgggccggaggagaccgactggtggtgggccgcgctgcacggccaggagggctacgtgccgcggaactacttcgggctgttccccagggtgaagcctcaaaggagtaaagtctagcaggatagaaggaggtttctgaggctgacagaaacaagcattcctgccttccctccagacctctccctctgttttttgctgcctttatctgcacccctcaccctgctggtggtggtccttgccaccggttctctgttctcctggaagtccagggaagaaggagggccccagccttaaatttagtaatctgccttagccttgggaggtctgggaagggctggaaatcactggggacaggaaaccacttccttttgccaaatcagatcccgtccaaagtgcctcccatgcctaccaccatcatcacatcccccagcaagccagccacctgcccagccgggcctgggatgggccaccacaccactggatattcctgggagtcactgctgacaccatctctcccagcagtcttggggtctgggtgggaaacattggtctctaccaggatccctgccccacctctccccaattaagtgccttcacacagcactggtttaatgtttataaacaaaatagagaaactggtttaatgtttataaacaaaatagagaaactttcgcttataaataaaagtagtttgcacagaaatgaaaaaaaaaaaaaaaaaaaaaaaa

SEQ ID NO: 2 - DNA, 1182 bp, Homo sapiens
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 3 - protein, 393 aa, Homo sapiens [sequence omitted]

SEQ ID NO: 4 - protein, 350 aa, Homo sapiens
MWMKDPVARPLSPTRLQPALPPEAQSVPELEEVARVLAEIPRPLKRRGSMEQAPAVALPPTHKKQYQQIISRLFHRHGGPGPGGRSQSCPPSLRDLRPGQGPLLLPHQLPFHRPAPSQSSPPEQPQSMEMRSVLRKAGSPRKARRARLNPLVLLLDAALTGELEVVQQAVKEMNDPSQPNEEGITALHNAICGANYSIVDFLITAGANVNSPDSHGWTPLHCAASCNDTVICMALVQHGAAIFATTLSDGATAFEKCDPYREGYADCATYLADVEQSMGLMNSGAVYALWDYSAEFGDELSFREGESVTVLRRDGPEETDWWWAALHGQEGYVPRNYFGLFPRVKPQRSK

SEQ ID NO: 5 - DNA, 4833 bp, Homo sapiens
gagccccgcatcccgccgcagctgccgcctcgccgcggccgggccggagagcacggcggcgggagcgcggccttaggaggcggccggagcggtgggcacagctcggcgcggagcgtcctgtcaggcggcggccgagggcgtcgcggactctccccgcgatgatgccgatgatattaactgttttcttgagcaacaatgaacagattttaacagaagttcctataacaccggaaacaacctgtcgagatgttgtagaattttgcaaggaacctggagaaggcagctgccatttagctgaagtgtggaggggaaatgaacgtcccataccctttgatcatatgatgtacgaacatcttcagatatggggtccacggagggaagaagtgaaatttttccttcgacacgaggactccccaactgagaacagtgaacaaggtggccgtcagacccaagagcaacgaactcagagaaatgtaataaatgtacctggagataaacgtactgaatatggggttgggtatccacgtgtagaacttaccctctcagagctccaagatatggcagctaggcaacagcagcagattgaaaatcagcagcagatgttggttgccaaggaacagcgtttacattttctaaagcaacaggagcgccgtcagcagcagtctatttctgaaaatgaaaagcttcagaaattgaaagaacgagttgaagcccaggagaacaagctgaagaaaattcgtgcaatgagaggacaagtcgactacagcaaaatcatgaacggcaatctgtctgctgaaatagaaaggttcagtgccatgttccaggaaaagaagcaggaagtacagactgcaattttaagggttgatcagcttagtcagcaattggaagatttaaagaaaggaaaactgaatgggttccagtcttacaatggcaaattgacgggaccagcggcggtggagttaaaaagactgtaccaagaactacagattcgtaaccaacttaaccaggaacaaaattcaaaacttcagcagcagaaggaactcttaaataagcgcaacatggaggtggccatgatggacaagcgaatcagtgaactgcgtgaacgtctctatgggaaaaaaattcagctgaaccgtgtgaatggcacgtcatcaccacagtcccctctgagcacatcgggcagggtcgctgctgtggggccttatatccaggttcccagtgccggaagctttcctgtgctgggggaccctataaagccccagtctctcagtattgcctcaaatgctgctcatggaagatccaaatccgctaatgatggaaactggccaacattaaaacagaattctagctcttccgtgaaaccagtgcaggtggccggtgcagactggaaggatccgagcgtggaggggtctgtcaagcagggcactgtctccagccagcctgtgcccttctcagcactgggacccacggagaagccgggcatcgagattggtaaagtgccacctcccatcccgggtgtaggcaagcagctgcctccaagctatgggacatacccaagtcctacacctctgggtcctgggtcgacaagctccctggaaaggaggaaggaaggcagcttgcccaggcccagtgcaggcctgccaagtcgacagaggcccaccctgctgcccgccacaggcagcaccccccagccaggctcctcacaacagattcagcagaggatttccgtaccgccaagtcccacgtacccgccagcgggaccacctgcatttccagctggggacagcaagcctgaactcccactgacagtggccattaggcctttcctggctgataaagggtcaaggccacagtctcccaggaaaggaccccagacagtgaattcaagttccatatactccatgtacctccagcaagccacaccacctaagaattaccagccggcagcacacagcgccttaaataagtcagttaaagcagtgtatggtaagcccgttttaccttcgggttcaacctctccatcgccgctgccgtttcttcacgggtcactgtccacgggcacaccacagcctcagccaccttcagaaagtactgagaaagagcctgagcaggatggccccgccgcccccgcagatggcagcaccgtggagagcctgccacggccactcagccccaccaagctcacgcccatcgtgcattcgccactgcgctaccagagtgatgcagacctggaggccctccgcaggaagctggccaacgcgccccggcccctgaaaaagcgcagctccatcacagagcccgagggccccggcgggcccaacatccagaagctgctgtaccagcgcttcaacaccctggccggtggcatggagggcacccctttctaccagcccagcccctcccaggacttcatgggcaccttggccgatgtggacaatggaaacaccaatgccaatggaaacctggaagagctcccccctgcccagcccacagccccactccccgctgagcctgccccgtcatcagatgccaatgataatgagttaccttcccccgaaccagaggagctcatctgtccccaaaccacccaccaaactgccgagccggcagaggacaataacaacaacgtggccacggtccccaccacggagcagatcccgagtcctgtggctgaggccccatctccaggggaagagcaggtccctccagcacctcttccccctgccagccaccctcctgccacctccacgaacaagcggaccaacttgaagaagcccaactcggagcggacggggcacgggctgagagtccggtttaaccccctggcactgctcctagacgcgtctctggaaggagagttcgatctggtgcagaggatcatctatgaggtggaagatcccagcaagcccaacgatgaagggatcaccccactgcacaacgccgtctgcgccggccaccatcacatcgtgaagttcctgctggattttggtgtcaacgtgaatgctgctgatagtgatggatggacgccgctgcactgcgctgcctcttgtaacagcgttcacctctgcaaacagctggtggagagtggtgccgccatttttgcctcaaccataagcgacattgaaactgctgcagacaagtgtgaggagatggaggaaggctacatccagtgctcccagtttctatatggggtgcaggaaaagctgggtgtgatgaacaaaggtgtggcgtatgctctgtgggactacgaggcccagaacagtgacgagctgtccttccacgaaggggacgccctcaccatcctgaggcgcaaggacgaaagcgagactgagtggtggtgggctcgccttggagaccgggagggctatgtgcccaaaaacctgctggggctgtatccacggatcaaaccccgacagcgaacactcgcctgaactccttttggagcaccgcatggtcttgccagctaccaggagccacttaagagattattgtgctgttttccaggaaagctgcagctagaaaatggtcttaatggtgctcactttagcagacagcgtccacaatgtgaatcctacagtttccaggtgaggccctttctccagtttgcccattaactgggagaggtactttcgcctccaaggactgaattttgccaattactataaatccaaataaatacccactttcaaaacacccacccctcttgccattaagaagtcccataacccccggttggttgccagtgaagacagaagctcttactgacttggccccgaggccatcaccccctccagcagtgaacactgtccgccgctgtgaggcctgctcccctgcgaccgccctgccccccgtcaccgaatcggacactcatcctttctcacacttcccacacatgatccttcttcccttcatcaccaaaggagcctctgtatggaaacatgtccagtgttgctgcccagtgtgtatgcctcccagtacccactctgctcggccgccttgggggttccgcttcctgttccagttcacctaaaggctgattgtgcaggcccagcactgtggctggactgccgcgccacgggcaccaggacccctaagaccaagtgacaactgggagagcctcagcatatactcttctcctccgatctcacagcctgtcatgctgctcagtgtggttctcacccctgcaagctcaaattcagttccctgaatggagtcaggtgctggaggccgtggcagcggagggtggttggggttggggctgggggtggactggtgtgagggcagaccagggccaggtagacggggctgtttggtgcctgaaggatggcagacgcctggtgtcaggaggggccgccaccaaggagcagcagctggggcagaggagctggggtcaggggccacccctctctgccgatctccctgcctgggctggctgtgaggccacctttgtcccaggcccagcctcaaggcaaggagggcgcttcactgaggtgtgaattgtacgtacaggctttttatataccaaaagtattttttgactagaccattcaaagctacccgaactatgttggaaatttttttttttctcattaaaatacaggcccttaggctctatttttcatgtatgagtcgtgtgtaatttatgtaaaaatgtgtgtacagactcactgatgcagcactgtagcccatcaccttggagcactgactgtacatagtgtggtgaagaaaagtgaacgcccttgtagagcagcccgaccacaggagcatggccgctgccagcccagacgctgctgacgctgtgtaaatgtgcacaataaacccgtctcaccccgg

SEQ ID NO: 6 - DNA, 4402 bp, Homo sapiens
gtcacgagcgtcgaagagacaaagccgcgtcagggggcccggccggggcgggggagcccggggcttgttggtgccccagcccgcgcggagggcccttcggacccgcgcgccgccgctgccgccgccgccgcctcgcaacaggtccgggcggcctcgctctccgctcccctcccccgcatccgcgaccctccggggcacctcagctcggccggggccgcagtctggccacccgcttccatgcggttcgggtccaagatgatgccgatgtttcttaccgtgtatctcagtaacaatgagcagcacttcacagaagttccagttactccagaaacaatatgcagagacgtggtggatctgtgcaaagaacccggcgagagtgattgccatttggctgaagtgtggtgtggctctgaacgtccagttgcggataatgagcgaatgtttgatgttcttcaacgatttggaagtcagaggaacgaagttcgcttcttccttcgtcatgaacgcccccctggcagggacattgtgagtggaccaagatctcaggatccaagtttaaaaagaaatggtgtaaaagttcctggtgaatatcgaagaaaggagaacggtgttaatagtcctaggatggatctgactcttgctgaacttcaggaaatggcatctcgccagcagcaacagattgaagcccagcaacaattgctggcaactaaggaacagcgcttaaagtttttgaaacaacaagatcagcgacaacagcaacaagttgctgagcaggagaaacttaaaaggctaaaagaaatagctgagaatcaggaagctaagctaaaaaaagtgagagcacttaaaggccacgtggaacagaagagactaagcaatgggaaacttgtggaggaaattgaacagatgaataatttgttccagcaaaaacagagggagctcgtcctggctgtgtcaaaagtagaagaactgaccaggcagctagagatgctcaagaacggcaggatcgacagccaccatgacaatcagtctgcagtggctgagcttgatcgcctctataaggagctgcagctaagaaacaaattgaatcaagagcagaatgccaagctacaacaacagagggagtgtttgaataagcgtaattcagaagtggcagtcatggataagcgtgttaatgagctgagggaccggctgtggaagaagaaggcagctctacagcaaaaagaaaatctaccagtttcatctgatggaaatcttccccagcaagccgcgtcagccccaagccgtgtggctgcagtaggtccctatatccagtcatctactatgcctcggatgccctcaaggcctgaattgctggtgaagccagccctgccggatggttccttggtcattcaggcttcagaggggccgatgaaaatacagacactgcccaacatgagatctggggctgcttcacaaactaaaggctctaaaatccatccagttggccctgattggagtccttcaaatgcagatcttttcccaagccaaggctctgcttctgtacctcaaagcactgggaatgctctggatcaagttgatgatggagaggttccgctgagggagaaagagaagaaagtgcgtccgttctcaatgtttgatgcagtagaccagtccaatgccccaccttcctttggtactctgaggaagaaccagagcagtgaagatatcttgcgggatgctcaggttgcaaataaaaatgtggctaaagtaccacctcctgttcctacaaaaccaaaacagattaatttgccttattttggacaaactaatcagccaccttcagacattaagccagacggaagttctcagcagttgtcaacagttgttccgtccatgggaactaaaccaaaaccagcagggcagcagccgagagtgctgctatctcccagcataccttcggttggccaagaccagaccctttctccaggttctaagcaagaaagtccacctgctgctgccgtccggccctttactccccagccttccaaagacaccttacttccacccttcagaaaaccccagaccgtggcagcaagttcaatatattccatgtatacgcaacagcaggcgccaggaaaaaacttccagcaggctgtgcagagcgcgttgaccaagactcataccagagggccacacttttcaagtgtatatggtaagcctgtaattgctgctgcccagaatcaacagcagcacccagagaacatttattccaatagccagggcaagcctggcagtccagaacctgaaacagagcctgtttcttcagttcaggagaaccatgaaaacgaaagaattcctcggccactcagcccaactaaattactgcctttcttatctaatccttaccgaaaccagagtgatgctgacctagaagccttacgaaagaaactgtctaacgcaccaaggcctctaaagaaacgtagttctattacagagccagagggtcctaatgggccaaatattcagaagcttttatatcagaggaccaccatagcggccatggagaccatctctgtcccatcatacccatccaagtcagcttctgtgactgccagctcagaaagcccagtagaaatccagaatccatatttacatgtggagcccgaaaaggaggtggtctctctggttcctgaatcattgtccccagaggatgtggggaatgccagtacagagaacagtgacatgccagctccttctccaggccttgattatgagcctgagggagtcccagacaacagcccaaatctccagaataacccagaagaaccaaatccagaggctccacatgtgcttgatgtgtacctggaggagtaccctccatacccacccccaccatacccatctggggagcctgaagggcccggagaagactcggtgagcatgcgcccgcctgaaatcaccgggcaggtctctctgcctcctggtaaaaggacaaacttgcgtaaaactggctcagagcgtatcgctcatggaatgagggtgaaattcaacccccttgctttactgctagattcgtctttggagggagaatttgaccttgtacagagaattatttatgaggttgatgacccaagcctgcccaatgatgaaggcatcacggctcttcacaatgctgtgtgtgcaggccacacagaaatcgttaagttcctggtacagtttggtgtaaatgtaaatgctgctgatagtgatggatggactccattacattgtgctgcctcatgtaacaacgtccaagtgtgtaagtttttggtggagtcaggagccgctgtgtttgccatgacctacagtgacatgcagactgctgcagataagtgcgaggaaatggaggaaggctacactcagtgctcccaatttctttatggagttcaggagaagatgggcataatgaataaaggagtcatttatgcgctttgggattatgaacctcagaatgatgatgagctgcccatgaaagaaggagactgcatgacaatcatccacagggaagacgaagatgaaatcgaatggtggtgggcgcgccttaatgataaggagggatatgttccacgtaacttgctgggactgtacccaagaattaaaccaagacaaaggagcttggcctgaaacttccacacagaattttagtcaatgaagaattaatctctgttaagaagaagtaatacgattatttttggcaaaaatttcacaagacttattttaatgacaatgtagcttgaaagcgatgaagaatgtctctagaagagaatgaaggattgaagaattcaccattagaggacatttagcgtgatgaaataaagcatctacgtcagcaggccatactgtgttggggcaaaggtgtcccgtgtagcactcagataagtatacagcgacaatcctgttttctacaagaatcctgtctagtaaataggatcatttattgggcagttgggaaatcagctctctgtcctgttgagtgttttcagcagctgctcctaaaccagtcctcctgccagaaaggaccagtgccgtcacatcgctgtctctgattgtccccggcaccagcaggccttggggctcactgaaggctcgaaggcactgcacaccttgtatattgtcagtgaagaacgttagttggttgtcagtgaacaataactttattatatgagtttttgtagcatcttaagaattatacatatgtttgaaatattgaaactaagctacagtaccagtaattagatgtagaatcttgtttgtaggctgaattttaatctgtatttattgtcttttgtatctcagaaattagaaacttgctacagacttacccgtaatatttgtcaagatcatagctgactttaaaaacagttgtaataaactttttgatgct

SEQ ID NO: 7 - protein, 1090 aa, Homo sapiens
MMPMILTVFLSNNEQILTEVPITPETTCRDVVEFCKEPGEGSCHLAEVWRGNERPIPFDHMMYEHLQIWGPRREEVKFFLRHEDSPTENSEQGGRQTQEQRTQRNVINVPGDKRTEYGVGNPRVELTLSELQDMAARQQQQIENQQQMLVAKEQRLHFLKQQERRQQQSISENEKLQKLKERVEAQENKLKKIRAMRGQVDYSKIMNGNLSAEIERFSAMFQEKKQEVQTAILRVDQLSQQLEDLKKGKLNGFQSYNGKLTGPAAVELKRLYQELQIRNQLNQEQNSKLQQQKELLNKRNMEVAMMDKRISELRERLYGKKIQLNRVNGTSSPQSPLSTSGRVAAVGPYIQVPSAGSFPVLGDPIKPQSLSIASNAAHGRSKSANDGNWPTLKQNSSSSVKPVQVAGADWKDPSVEGSVKQGTVSSQPVPFSALGPTEKPGIEIGKVPPPIPGVGKQLPPSYGTYPSPTPLGPGSTSSLERRKEGSLPRPSAGLPSRQRPTLLPATGSTPQPGSSQQIQQRISVPPSPTYPPAGPPAFPAGDSKPELPLTVAIRPFLADKGSRPQSPRKGPQTVNSSSIYSMYLQQATPPKNYQPAAHSALNKSVKAVYGKPVLPSGSTSPSPLPFLHGSLSTGTPQPQPPSESTEKEPEQDGPAAPADGSTVESLPRPLSPTKLTPIVHSPLRYQSDADLEALRRKLANAPRPLKKRSSITEPEGPGGPNIQKLLYQRFNTLAGGMEGTPFYQPSPSQDFMGTLADVDNGNTNANGNLEELPPAQPTAPLPAEPAPSSDANDNELPSPEPEELICPQTTHQTAEPAEDNNNNVATVPTTEQIPSPVAEAPSPGEEQVPPAPLPPASHPPATSTNKRTNLKKPNSERTGHGLRVRFNPLALLLDASLEGEFDLVQRIIYEVEDPSKPNDEGITPLHNAVCAGHHHIVKFLLDFGVNVNAADSDGWTPLHCAASCNSVHLCKQLVESGAAIFASTISDIETAADKCEEMEEGYIQCSQFLYGVQEKLGVMNKGVAYALWDYEAQNSDELSFHEGDALTILRRKDESETEWWWARLGDREGYVPKNLLGLYPRIKPRQRTLA

SEQ ID NO: 8 - protein, 1128 aa, Homo sapiens
MMPMFLTVYLSNNEQHFTEVPVTPETICRDVVDLCKEPGESDCHLAEVWCGSERPVADNERMFDVLQRFGSQRNEVRFFLRHERPPGRDIVSGPRSQDPSLKRNGVKVPGEYRRKENGVNSPRMDLTLAELQEMASRQQQQIEAQQQLLATKEQRLKFLKQQDQRQQQQVAEQEKLKRLKEIAENQEAKLKKVRALKGHVEQKRLSNGKLVEEIEQMNNLFQQKQRELVLAVSKVEELTRQLEMLKNGRIDSHHDNQSAVAELDRLYKELQLRNKLNQEQNAKLQQQRECLNKRNSEVAVMDKRVNELRDRLWKKKAALQQKENLPVSSDGNLPQQAASAPSRVAAVGPYIQSSTMPRMPSRPELLVKPALPDGSLVIQASEGPMKIQTLPNMRSGAASQTKGSKIHPVGPDWSPSNADLFPSQGSASVPQSTGNALDQVDDGEVPLREKEKKVRPFSMFDAVDQSNAPPSFGTLRKNQSSEDILRDAQVANKNVAKVPPPVPTKPKQINLPYFGQTNQPPSDIKPDGSSQQLSTVVPSMGTKPKPAGQQPRVLLSPSIPSVGQDQTLSPGSKQESPPAAAVRPFTPQPSKDTLLPPFRKPQTVAASSIYSMYTQQQAPGKNFQQAVQSALTKTHTRGPHFSSVYGKPVIAAAQNQQQHPENIYSNSQGKPGSPEPETEPVSSVQENHENERIPRPLSPTKLLPFLSNPYRNQSDADLEALRKKLSNAPRPLKKRSSITEPEGPNGPNIQKLLYQRTTIAAMETISVPSYPSKSASVTASSESPVEIQNPYLHVEPEKEVVSLVPESLSPEDVGNASTENSDMPAPSPGLDYEPEGVPDNSPNLQNNPEEPNPEAPHVLDVYLEEYPPYPPPPYPSGEPEGPGEDSVSMRPPEITGQVSLPPGKRTNLRKTGSERIAHGMRVKFNPLALLLDSSLEGEFDLVQRIIYEVDDPSLPNDEGITALHNAVCAGHTEIVKFLVQFGVNVNAADSDGWTPLHCAASCNNVQVCKFLVESGAAVFAMTYSDMQTAADKCEEMEEGYTQCSQFLYGVQEKMGIMNKGVIYALWDYEPQNDDELPMKEGDCMTIIHREDEDEIEWWWARLNDKEGYVPRNLLGLYPRIKPRQRSLA

SEQ ID NO: 9 - DNA, 64 bp, Homo sapiens
gatcccctgtcaactcccccgacagcttcaagagagctgtcgggggagttgacatttttggaaa

SEQ ID NO: 10 - DNA, 64 bp, Homo sapiens
agcttttccaaaaatgtcaactcccccgacagctctcttgaagctgtcgggggagttgacaggg

SEQ ID NO: 11 - DNA, 28 bp, Homo sapiens
ccagcaagccacaccacctaagaattac

SEQ ID NO: 12 - DNA, 26 bp, Homo sapiens
tgaacccgaaggtaaaacgggcttac

SEQ ID NO: 13 - DNA, 20 bp, Homo sapiens
gaagactcggtgagcatgcg

SEQ ID NO: 14 - DNA, 20 bp, Homo sapiens
gcgatacgctctgagccagt

SEQ ID NO: 15 - DNA, 22 bp, Homo sapiens
caggcggtgaaggagatgaacg

SEQ ID NO: 16 - DNA, 25 bp, Homo sapiens
aaatccacgatagagtagttggcgc

SEQ ID NO: 17 - DNA, 22 bp, Homo sapiens
ccggcagcacacagcgccttaa

SEQ ID NO: 18 - DNA, 26 bp, Homo sapiens
ccgcctgaaatcaccggggcaggtct

SEQ ID NO: 19 - DNA, 20 bp, Homo sapiens
cccgagccagcccaacgagg